(19) United States
(12) Patent Application Publication
(10) Pub. No.: US 2022/0177881 A1
(43) Pub. Date: Jun. 9, 2022

(54) ENGINEERED EXOSOMES FOR TARGETED DELIVERY

(71) Applicant: THE TRUSTEES OF COMUMBIA UNIVERSITY IN THE CITY OF NEW YORK, New York, NY (US)

(72) Inventor: Fatemeh MOMEN-HERAVI, Teaneck, NJ (US)

(21) Appl. No.: 17/681,177

(22) Filed: Feb. 25, 2022

Related U.S. Application Data
(63) Continuation of application No. PCT/US2020/047894, filed on Aug. 26, 2020.
(60) Provisional application No. 62/892,231, filed on Aug. 27, 2019.
(60) Provisional application No. 62/923,724, filed on Oct. 21, 2019.

(51) Int. Cl. C12N 15/11; C12N 9/22; C12N 15/90; A61K 31/713; A61K 9/51

(52) U.S. Cl. CPC C12N 15/111; C12N 9/22; C12N 15/90; C12N 2320/32; A61K 9/51; C12N 2310/14; C12N 2310/20; A61K 31/713

(57) ABSTRACT

The present disclosure provides for an engineered exosome or extracellular vesicle, wherein the engineered exosome or extracellular vesicle is substantially devoid of endogenous nucleic acids and can comprise at least one targeting moiety and/or at least one payload or cargo. The payload or cargo can be a diagnostic agent or a therapeutic agent such as exogenous nucleic acids and/or a CRISPR/Cas system for gene editing. The engineered exosomes can be used to treat disease.

### CROSS-REFERENCE TO OTHER APPLICATIONS

The present application is a continuation of PCT/US2020/047894, filed Aug. 26, 2020, which claims priority to U.S. Patent Applications Ser. No. 62/892,231 filed Aug. 27, 2019, and 62/923,724 filed Oct. 21, 2019.

### BACKGROUND

In 2013, the discovery of CRISPR/Cas revolutionized the field of genome editing. CRISPR/Cas components are a part of the bacterial immune system and are able to induce double-strand DNA breaks in the genome which are resolved by endogenous DNA repair mechanisms (Kick et al. 2017). The CRISPR/Cas system can be repurposed for genome editing in mammalian cells (Cong et al. 2013). Cas proteins can be programmed to target nearly any gene in the genome by synthesizing a guide RNA (gRNA) molecule complementary to the target sequence (Wang et al. 2017; Jinek et al. 2012). After the introduction of a double strand break, the broken DNA ends are recognized by proteins belonging to the DNA repair machinery. The design flexibility of the CRISPR system has driven the rapid adoption of this method for an array of genome editing applications in the laboratory.

While CRISPR/Cas mediated therapeutic gene knockout and corrections have many potential applications, their practical execution is not straight forward. One of the main challenges of using CRISPR/Cas for treatment of human diseases is delivering the system at the targeted cells and organs with high efficiency and thereby avoiding gene edits in non-target cells. Multiple components need to be delivered into the nuclei of target cells at the right dose and at the right time for the desired therapeutic effects. In vivo, CRISPR/Cas can be delivered locally or systemically and there are several formats in which the gRNA and Cas proteins can be delivered to the cell to achieve therapeutic gene editing in different forms including: plasmids or viral vectors that carry Cas9 and gRNA genes in gene-based delivery; Cas mRNA and a synthetic gRNA in RNA-based delivery; and Cas protein and a synthetic gRNA in protein-based delivery.

Although a number of CRISPR delivery platforms have been created so far, the effective delivery of multiple CRISPR components in vivo into host cells still remains a major challenge. Adenovirus (AV) is an efficient transducing agent used for CRISPR/Cas-mediated genome editing, however, this vector can elicit a significant immune response in the host (Wang et al. 2004). Lentiviral vectors are at present the most widely used viral vectors for clinical therapy, however, they integrate into the genome which makes them counterproductive for gene editing purposes. Long lasting expression of Cas protein is considered to be unfavorable for the on-target/off-target ratio of indel formation (Banaszynski et al. 2006). Viral vectors are currently limited in terms of cargo carrying capacity and tropism.

Non-viral synthetic vectors which also can be used as delivery methods lack tissue tropism and targeted cell-specific and organ-specific delivery, unless targeting moieties such as peptides or antibodies are added (Peer et al. 2007). Additionally, synthetic delivery vectors have disadvantages such as problems with biocompatibility and toxicity, immunogenic potential, and problems with therapeutic cargo release (Wilbie et al. 2019). This targeting is particularly difficult to achieve, as incorporation of additional biomolecules to a delivery vector alongside the CRISPR components complicates the packaging (Mout et al. 2017).

Another major potential barrier for in vivo delivery is the immune response that may be raised in response to the large amounts of virus necessary for treatment, this phenomenon is not unique to genome editing and is observed with other virus-based gene therapies.

It is also difficult, with this mode of therapy, to control the distribution and the dosage of genome editing nucleases in vivo, leading to off-target mutation profiles that may be hard to predict (Banaszynski et al. 2006; Wilbie et al. 2019).

In vitro, cell lines can be transfected with lentivirus carrying genetically encoded Cas and gRNA, or these components can be introduced to the cells by transfection reagents or electroporation. However, these methods are not suitable for in vivo delivery since there is a need for a specific robust and targeted delivery of both Cas9 and gRNA to exert the therapeutic benefit. Moreover, delivery should be in the proper therapeutic dosage, in the desired window of time, and must be specific to the cell and tissue of interest to prevent off-target gene editing events.

Another challenge is the difficulty of transporting Cas and gRNA across the cell membranes, compounded by the possibility for the CRISPR complex being degraded by proteases, RNases, or lysosomes, which can elicit an immune response. A well-designed delivery vehicle which enables targeted delivery is needed to mitigate these challenges.

Exosomes are nanosized (50-150 nm) membrane bounded vesicles secreted by almost all types of cells in the cellular microenvironment and are found in biofluids (Momen-Heravi et al. 2013; Momen-Heravi et al. 2018; Lotvall et al. 2014). They naturally carry biomacromolecules—including different RNAs (mRNAs, regulatory miRNAs), DNAs, lipids, and proteins—and can efficiently deliver their cargoes to recipient cells, eliciting functions, and mediating cellular communications (Thery et al. 2002). Previous work by this group and by others have shown the advantages of using exosomes for drug delivery: 1) exosomes are small and have a high efficiency for delivery due to their similarity to cell membranes; 2) exosomes are biocompatible, non-immunogenic, and non-toxic, even in repeated in vivo injections (Momen-Heravi et al. 2014); 3) exosomes are stable even after several freeze and thaw cycles, and their lipid bilayer protects the protein and RNA cargoes from enzymes such as proteases and RNases (Momen-Heravi et al. 2018); 4) exosomes have slightly negative zeta potential, leading to long circulation (Malhortra et al. 2016); and 5) exosomes also exhibit an increased capacity to escape degradation or clearance by immune system (Hood 2016). Although exogenous exosomes have been used for delivery of RNA interference (RNAi), miRNAs and mRNA by this group and others (Momen-Heravi et al. 2014; Bukong et al. 2014; Bala et al. 2015), the successful modification of exosomes for targeted delivery of CRISPR/Cas system as a scalable, target specific, efficient treatment modality, without unwanted biological effects, has not been shown and requires several innovative approaches.

Shown herein are engineered exosomes which contain minimal endogenous nucleic acids and are targeted to particular cells, tissues and organs and can be further engineered to contain a cargo or payload including but not limited to nucleic acids and/or CRISPR-Cas systems.

### SUMMARY

In one embodiment, the disclosure provides for an engineered exosome or extracellular vesicle, wherein the engineered exosome or extracellular vesicle is substantially devoid of endogenous nucleic acids. In a further embodiment, the disclosure provides that the engineered exosome or extracellular vesicle is further engineered to express at least one targeting moiety on its surface. In further embodiments, the engineered exosome or extracellular vesicle is further engineered to comprise or contain at least one cargo or payload.

A further embodiment of the current disclosure provides for an engineered exosome or extracellular vesicle comprising at least one targeting moiety expressed on the surface of the exosome or extracellular vesicle and at least one cargo or payload, wherein the engineered exosome or extracellular vesicle is substantially devoid of endogenous nucleic acids.

In a further embodiment, the current disclosure provides for an engineered exosome or extracellular vesicle comprising at least one targeting moiety or a therapeutic molecule expressed on the surface of the exosome or extracellular vesicle and at least one cargo or payload, wherein the engineered exosome or extracellular vesicle is substantially devoid of endogenous nucleic acids.

In some embodiments, the exosome or the extracellular vesicle is derived from cells or tissue including but not limited to bone marrow, red blood cells, immune cells, human monocytes and macrophages, tumor cells, epithelial cells and stem cells from primary cells or autologous cells. In some embodiments, the exosome or the extracellular vesicle is derived from cultured cells.

In some embodiments, the targeting moiety targets a specific cell, tissue or organ. In some embodiments, the targeting moiety targets specific tissue, including but not limited to lung tissue, spleen tissue, digestive organ tissue, liver tissue, kidney tissue or brain tissue. In some embodiments, the targeting moiety targets a specific cell including but not limited to epithelial cells, tumor cells, fibroblast, monocytes, macrophages, dendritic cells, natural killer cells, T cells, and B cells.

In some embodiments, the targeting moiety comprises an integrin, a laminin, an antibody, an antibody fragment, a receptor, a component of extracellular matrix, and/or a peptide. In some embodiments, the targeting moiety is an integrin. In some embodiments, the integrin targets liver tissue. In some embodiments, the integrin is αvβ5. In some embodiments, the integrin targets lung tissue. In some embodiments, the integrin is α6β4. In some embodiments, the integrin is α6β4. In some embodiments, the targeting moiety comprises a fusion protein. In some embodiments, the fusion protein comprises the targeting moiety fused with a ubiquitous marker for exosomes. In some embodiments, the marker is CD63, CD81 or CD9.

In some embodiments, the therapeutic molecule is an antibody, a peptide, a cytokine, a signaling molecule or a small molecule. In some embodiments, the therapeutic molecule comprises a fusion protein. In some embodiments, the fusion protein comprises the therapeutic molecule fused with a ubiquitous marker for exosomes. In some embodiments, the marker is CD63, CD81 or CD9.

In some embodiments, the engineered exosome or extracellular vesicle comprises or contains a cargo or payload. In some embodiments, the cargo or payload is a therapeutic agent. In some embodiments, the cargo or payload is a diagnostic agent.

The cargo or payload of the present engineered exosome or extracellular vesicle includes, but is not limited to, a nucleic acid (DNA, RNA, etc.), a protein, a peptide, a polypeptide, and/or a small molecule.

Proteins or polypeptides include, but are not limited to, a nuclease (an endonuclease or an exonuclease), a peptide, an antibody or a fragment thereof, or combinations thereof.

Nucleic acids include DNA or RNA. DNA includes a single-stranded DNA (ssDNA), a double-stranded DNA (dsDNA), a donor/template DNA, a DNA encoding one or more RNAs, cDNA or combinations thereof. RNA includes a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, or combinations thereof.

The cargo or payload may be a DNA digesting agent.

Cargo or payloads also include but are not limited to and engineered Clustered Regularly Interspaced Short Palindromic Repeats (CRISPR)-CRISPR associated (Cas) (CRISPR-Cas) system. In some embodiments, the CRISPR-Cas system comprises Cas9, CAS13a, CAS13b, CAS13c, CAS13d, Cas12a, SiT-Cas12a, Cpf1, c2c1, C2c2, C2c3, dCas9 or any of those CAS proteins fused to other proteins. In some embodiments, the payload or cargo also includes a sgRNA. In some embodiments, the sgRNA forms a Cas-gRNA ribonucleoprotein complex with the Cas protein. In some embodiments, the cargo or payload includes a donor DNA. In some embodiments, the cargo or payload includes a pegRNA.

The disclosure also provides for compositions, including pharmaceutical compositions comprising any of the engineered exosomes or extracellular vesicles described herein. The disclosure also provides for engineered exosomes or extracellular vesicles described herein for use in treating a disease, and methods of treatment of disease using any of the engineered exosomes or extracellular vesicles described herein. Diseases for which the engineered exosomes or extracellular vesicles can be used to treat include but are not limited to cancer and cystic fibrosis.

In particular, in some embodiments, when the exosome or the extracellular vesicle is used for treating cystic fibrosis, the gRNA can target a sequence corresponding to codon 489 on the transmembrane conductance regulator (CFTR) protein. In this embodiment, the donor DNA is a single stranded DNA of about 20 nucleotides. In some embodiments, the exosome or the extracellular vesicle is used for treating non-small cell lung cancer, and a prime editing CRISPR system is used.

The disclosure also provides for kits.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 is a graph of the total RNA in exosomes produced by post-transcriptional silencing of hnRNPA2B1 (approximately by 85%), Dorsha silencing (approximately by 70%), and Alix silencing (approximately by 80%). In each case, there was a significant reduction of total RNA in the treated exosomes. * indicates statistically significant, (p-value<0.001) (n=4 per group).

FIG. 2 show the alpha 6 beta 4 (α6β4) integrin cloned with promotor of CD63. FIG. 2A is a schematic of the construct. FIG. 2B is a Western blot demonstrating expression of beta4 on engineered exosomes.

FIG. 3 shows that the engineered exosomes are taken up by cells. FIG. 3A is a graph showing results of PE-labeled engineered exosomes taken up by primary macrophages in a dose-dependent manner (left panel: 50 μg of exosomal proteins; right panel: 100 μg of exosomal proteins). The uptake was qualified by flow-cytometry based on the percentage of PE-positive cells. FIG. 3B are images of engineered exosomes labeled with PKH26 fluorescent dye or Di-8 annepps dye and cocultured with cells and imaged 1 hour after co-culture.

FIG. 4 shows that engineered exosomes loaded with exogenous nucleic acid are taken up by cells. FIG. 4A is a graph showing engineered exosomes loaded with Cel-miR-39-3P and co-cultured with HCC827 cells. Levels of Cel-miR-39-3P were quantified by quantitative real time polymerase chain reaction 1 hour after co-culture (n=4 in each group). FIG. 4B is a graph of engineered exosomes loaded with microRNA-34a mimic and co-cultured with HCC827 cells. Levels of microRNA-34a were quantified by quantitative real time polymerase chain reaction 1 hour after co-culture. (n=3 in each group). FIG. 4C is a graph of engineered exosomes loaded with microRNA-34a inhibitor and co-cultured with HCC827 cells. Levels of microRNA-34a were quantified by quantitative real time polymerase chain reaction 1 hour after co-culture (n=3 in each group). RNU48 was used to normalize the Ct values between the samples. * indicates p value <0.05.

FIG. 5 shows results of TaqMan Pri-miRNA assays used to quantify pri-microRNA-34a in HCC827 cells indicating no change in the primary transcript which corroborate horizontal transfer of microRNA-34a by exosomes as presented in FIG. 4. GAPDH was used as to normalize the Ct values between the samples. (n=3 per group).

FIG. 6 is a graph of the results of engineered exosomes loaded with siRNA against STAT3 and co-cultured with HCC827 cells. Levels of STAT3 were quantified by quantitative real time polymerase chain reaction 36 hours after co-culture. mRNA level was normalized against 18s. * indicates p value <0.05 (n=5 in each group).

FIG. 7 shows the engineered exosomes were successfully delivered in vivo. FIG. 7A is a graph showing results of Cel-miR-39-3p loaded exosomes injected IV into BALB/c mice. Levels of Cel-miR-39-3p were quantified in lung 10 minutes after injection. A significant increase the miRNA in the mice which received the Cel-miR-39-3p indicates the successful delivery of that microRNA to the lungs (n=5 per group). FIG. 7B is an image of fluorescently labeled engineered exosomes with targeted moieties for lung reinjected into mice IV and imaged with IVIS spectrum imaging. Fluorescent signal in the lung indicates successful delivery of those exosomes to the lung.

FIG. 8 is a Western blot of THP1 cells engineered to express endogenous CAS9 and package the CAS9 into the exosomes. CAS9 was expressed in the exosomes derived from engineered cell lines and engineered cells line but not in the non-engineered exosomes (THP1 exosomes).

FIG. 9 shows that the engineered exosomes expressing CAS are capable of actively editing genes. FIG. 9A are the results of a T7 endonuclease assay (mismatch assay) to monitor genome editing at the EXM-1 gene in human macrophages. Presence of a double band indicates indel formation and successful gene editing by engineered CAS expressing exosomes. FIG. 9B is a graph of the levels of modified read (indel formation) quantified using next generation sequencing. Groups which included engineered exosomes for CRISPR/CAS delivery resulted in a more efficient gene editing compared to other methods.

FIG. 10 is a Western blot showing that a Flag sequence was inserted using CAS9+ssDNA in form of plasmid with transfection or delivered by Cas expressing engineered exosomes. Insertion was assessed by PCR using a forward primer in the flag-sequence and a reverse primer using primers flanking the Flag sequence (Insertion PCR assay). Cells were pretreated with DCLREIC siRNA and XRCC5 siRNA 24 hours before gene editing.

FIG. 11 is a graph showing the cytokine expression levels as measured by qPCR in untreated or Cas9 expressing engineered exosomes (CAS9exo-treated) treated human macrophages upon LPS stimulation. No significant increase in cytokine level was found in the Cas9 expressing engineered exosomes.

FIG. 12 is a schematic experiment design for using engineered exosomes with CRISPR/Cas machinery and targeted for lung delivery for treatment of cystic fibrosis.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “substantially free” or “substantially devoid” is used operationally, in the context of analytical testing of the exosome or extracellular vesicle. An exosome or extracellular vesicle substantially devoid or free of endogenous nucleic acids contains no greater than 10%, no greater than 8%, no greater than 5%, no greater than 2%, or no greater than 1%, of endogenous nucleic acids.

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. “Substantially complementary” refers to a degree of complementarity that is about or more than about 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides (e.g., contiguous nucleotides), or refers to two nucleic acids that hybridize under stringent conditions.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally occurring as a whole. Parts of the vectors can be naturally occurring. The non-naturally occurring recombinant expression vectors of the present disclosure can comprise any type of nucleotides, including, but not limited to DNA and RNA, which can be single-stranded or double-stranded, synthesized or obtained in part from natural sources, and which can contain natural, non-natural or altered nucleotides.

“Transfection,” “transformation,” or “transduction,” as used herein, refer to the introduction of one or more exogenous polynucleotides into a host cell by using physical or chemical methods.

“Antibody,” “fragment of an antibody,” “antibody fragment,” “functional fragment of an antibody,” or “antigen-binding portion” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide linked Fv, Fc, or variants and/or mixtures. The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

The term “inhibition” when used in reference to gene expression or function of a protein or polypeptide refers to a decrease in the level of gene expression or function of the protein or polypeptide, where the inhibition is a result of interference with gene expression or function. The inhibition may be complete, in which case there is no detectable expression or function, or it may be partial. Partial inhibition can range from near complete inhibition to a near absence of inhibition.

As used herein, the term “therapeutically effective amount” is an amount sufficient to treat a specified disorder or disease or alternatively to obtain a pharmacological response treating a disorder or disease.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the engineered exosome or extracellular vesicle) and does not negatively affect the subject to which the composition(s) are administered. The pharmaceutical compositions may comprise pharmaceutically acceptable carriers, excipients, or stabilizers in the form of lyophilized formations or aqueous solutions.

It is to be understood, although not always explicitly stated, that all numerical designations may be preceded by the term “about”. Where particular values are described in the application and claims, unless otherwise stated, the term “about” meaning within an acceptable error range for the particular value should be assumed. As used herein, the term “about” refers to a particular value ±10%.

### Extracellular Vesicles and Exosomes

Extracellular vesicles are membrane enclosed vesicles released by cells. Their primary constituents are lipids, proteins and nucleic acids. They are composed of a lipid-protein bilayer encapsulating an aqueous core comprising nucleic acids and soluble proteins. Extracellular vesicles include, but are not limited to, exosomes, shedding vesicles, microvesicles, small vesicles, large vesicles, microparticles, and apoptotic bodies, based on their size, cellular origin and formation mechanism. Exosomes are formed by inward budding of late endosomes forming multivesicular bodies (MVB) which then fuse with the limiting membrane of the cell concomitantly releasing the exosomes. Shedding vesicles are formed by outward budding of the limiting cell membrane followed by fusion. When a cell undergoes apoptosis, the cell disintegrates and divides its cellular content in different membrane enclosed vesicles termed apoptotic bodies. Non-limiting examples of extracellular vesicles include circulating extracellular vesicles, beta cell extracellular vesicles, islet cell extracellular vesicles, exosomes and apoptotic bodies, and combinations thereof.

Large extracellular vesicles can range from about 5 μm to about 12 μm in diameter. Apoptotic bodies can range from about 1 μm to about 5 μm in diameter. Microvesicles can range from about 100 nm to about 1 μm in diameter. Exosomes can range from about 30 nm to about 150 nm, from about 30 nm to about 100 nm, or from about 50 nm to about 150 nm in diameter or from about 50 nm to about 200 nm.

Extracellular vesicles or exosomes may be isolated or derived from bone marrow, red blood cells, tumor cells, immune cells, epithelial cells, fibroblasts, or stem cells. Extracellular vesicles or exosomes may be isolated or derived from B cells, T cells, monocytes, or macrophages. In one embodiment, extracellular vesicles or exosomes to be taken up by a specific type of cells (e.g., monocytes/macrophages) are isolated or derived from the same type of cells (e.g., monocytes/macrophages).

Extracellular vesicles or exosomes may be isolated or derived from a body fluid. For example, the body fluids can include, but are not limited to, serum, plasma, blood, whole blood and derivatives thereof, urine, tears, saliva, sweat, cerebrospinal fluid (CSF), oral mucus, vaginal mucus, seminal plasma, semen, prostatic fluid, excreta, ascites, lymph, bile, breast milk and amniotic fluid.

Extracellular vesicles or exosomes may be isolated or derived from cultured cells.

Methods for isolating extracellular vesicles include size separation methods such as centrifugation. In one embodiment, isolating various components of extracellular vesicles may be through an isolation method including sequential centrifugation. The method may include centrifuging a sample at 800 g for a desired amount of time, collecting the pellet containing cells and cellular debris and (first) supernatant, centrifuging the (first) supernatant at 2,000 g for a desired time, collecting the pellet containing large extracellular vesicles and apoptotic bodies and (second) supernatant. The sequential centrifugation method can further include centrifuging the (second) supernatant at 10,000 g, collecting the pellet containing microvesicles and (third) supernatant. The sequential centrifugation method can further include centrifuging the (third) supernatant at 100,000 g, collecting the pellet containing exosomes (ranging from about 30 nm to about 200 nm in diameter) and (fourth) supernatant. The sequential centrifugation method can further include washing each of the pellets including the extracellular vesicles (e.g., large extracellular vesicles and apoptotic bodies, microvesicles, and exosomes) such as in phosphate buffered saline followed by centrifugation at the appropriate gravitational force and collecting the pellet containing the extracellular vesicles. Isolation, purity, concentration, size, size distribution, and combinations thereof of the extracellular vesicles following each centrifugation step can be confirmed using methods such as nanoparticle tracking, transmission electron microscopy, immunoblotting, and combinations thereof. Nanoparticle tracking (NTA) to analyze extracellular vesicles such as for concentration and size can be performed by dynamic light scattering using commercially available instruments such as ZETAVIEW (commercially available from ParticleMetrix, Meerbusch, Germany). Following isolation, the method can further include detecting an extracellular vesicle marker.

Methods for isolating extracellular vesicles also include using commercially available reagents such as, for example, EXOQUICK TC reagent (commercially available from System Biosciences, Palo Alto, Calif.).

Exosomes are small vesicular bodies that are secreted from cells into the cellular microenvironment and biofluids and can enter both neighboring cells and the systemic circulation. Exosomes are actively assembled from intracellular multivesicular bodies (MVBs) by the endosomal sorting complex required for transport (ESCRT) machinery. Exosomes contain various molecular constituents of their cell of origin, including, but not limited to, proteins, RNA (such as mRNA, miRNA, etc.), lipids and DNA.

Exosome may be isolated by any suitable techniques, including ultracentrifugation, micro-filtration, size-exclusion chromatography etc. or a combination thereof. Exosome can be isolated using a combination of techniques based on both physical (e.g., size, density) and biochemical parameters (e.g., presence/absence of certain proteins involved in their biogenesis). In certain embodiments, exosomes are isolated using a kit. In one embodiment, exosomes are isolated using the Total Exosome Isolation Kit and/or the Total Exosome Isolation Reagent from Invitrogen.

Following isolation, the method can further include detecting an extracellular vesicle marker of the extracellular vesicle.

Extracellular vesicle or exosome markers include CD9, CD63, CD81, LAPM1, TSG101, and combinations thereof.

### Exosomes or Extracellular Vesicles Substantially Devoid of Endogenous Nucleic Acids

Endogenous nucleic acids of an exosome or extracellular vesicle can include DNA or RNA. RNAs include messenger RNA (mRNA), microRNA (miRNA), long intergenic non-coding RNA (lincRNA), long non-coding RNA (lncRNA), non-coding RNA (ncRNA), non-messenger RNA (nmRNA), small RNA (sRNA), small non-messenger RNA (smnRNA), DNA damage response RNA (DD RNA), extracellular RNA (exRNA), small nuclear RNA (snRNA), small nucleolar RNA (snoRNA), and precursor messenger RNA (pre-mRNA).

To make exosomes or extracellular vesicles biologically safe and to eliminate the unwanted side effects associated with the endogenous exosomal cargo, exosomes or extracellular vesicles can be engineered to be substantially free or devoid of endogenous nucleic acids. This can result in minimal off-target effects.

To prepare exosomes or extracellular vesicles substantially devoid of endogenous nucleic acids, one or more players in sorting or loading nucleic acids into exosomes or extracellular vesicles may be downregulated or inhibited. These players include but are not limited to heterogeneous nuclear ribonucleoprotein A2B1 (hnRNPA2B1), Dorsha, Alix, major vault protein (MVP), Exportin 1 and Exportin 5. The protein hnRNPA2B1 specifically binds exosomal RNA through the recognition of specific motifs, controlling their loading into exosomes. Villarroya-Beltri et al. (2013). Thus, by knocking down hnRNPA2B1, the key player in sorting RNAs into exosomes, exosomes can be engineered to be substantially free or devoid of endogenous nucleic acids. Dorsha functions as the initiator of microRNA biogenesis by cleaving pri-miRNA to mature forms of microRNA. Alix mediates nucleic acid loading into the exosomes. Han et al. 2004; Iavello et al. 2016.

The amount/level and/or activity of hnRNPA2B1, Dorhsa, and/or Alix, etc. may be downregulated or otherwise decreased or suppressed. The mechanism of inhibition may be at the genetic level (e.g., interference with or inhibit expression, transcription or translation, etc.) or at the protein level (e.g., binding, competition, etc.).

### Cargo and Payload

The cargo or payload of the present engineered exosome or extracellular vesicle includes but is not limited to therapeutic and diagnostic agents.

The cargo or payload of the present engineered exosome or extracellular vesicle includes, but is not limited to, a nucleic acid (DNA, RNA, etc.), a protein or polypeptide, and/or a small molecule.

Proteins or polypeptides include, but are not limited to, a nuclease (an endonuclease or an exonuclease), an antibody or a fragment thereof, or combinations thereof.

Nucleic acids include DNA or RNA. DNA includes a single-stranded DNA (ssDNA), a double-stranded DNA (dsDNA), a donor/template DNA, a DNA encoding one or more RNAs, cDNA, or combinations thereof. RNA includes a sgRNA, a guide RNA (gRNA), prime editing guide RNA (pegRNA), a microRNA (miRNA) inhibitor, a miRNA mimic, a small interfering RNA (siRNA), small synthetic RNA, an antisense oligonucleotide, a short hairpin RNA (shRNA), a double-stranded RNA (dsRNA), an antisense RNA, a ribozyme, or combinations thereof. The cargo or payload may be a DNA digesting agent.

In certain embodiments, the nucleic acid (e.g., gRNA, sgRNA, etc.) may contain a nuclear import signal (NLS) which may increase the efficacy of editing in the nucleus. An NLS is an amino acid sequence that tags a protein for import into the cell nucleus by nuclear transport. In one embodiment, the NLS is the Chelsky sequence motif of K-K/R-x-K/R (Lys-Lys/Arg-x-Lys/Arg) which leads to nuclear localization via importin a. In another embodiment, the NLS is a bipartite NLS of nucleoplasmin, or other NLS sequences such as c-Myc (PAAKRVKLD) and TUS-protein (KLKIKRPVK).

As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules.

The term “DNA digesting agent” refers to an agent that is capable of cleaving bonds (i.e., phosphodiester bonds) between the nucleotide subunits of nucleic acids.

In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyzes the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

### Endonucleases

Non-limiting examples of the endonucleases include a zinc finger nuclease (ZFN), a ZFN dimer, a ZFNickase, a transcription activator-like effector nuclease (TALEN), a meganuclease, or an RNA-guided DNA endonuclease (e.g., CRISPR/Cas systems). Meganucleases include endonucleases in the LAGLIDADG and PI-Sce family.

TALENs are composed of a TAL effector domain that binds to a specific nucleotide sequence and an endonuclease domain that catalyzes a double strand break at the target site (PCT Patent Publication No. WO2011072246; Miller et al., Nat. Biotechnol. 29, 143-148 (2011); Cermak et al., Nucleic Acid Res. 39, e82 (2011)). Sequence-specific endonucleases may be modular in nature, and DNA binding specificity is obtained by arranging one or more modules (Bibikova et al., Mol. Cell. Biol. 21, 289-297 (2001); Boch et al., Science 326, 1509-1512 (2009)).

ZFNs can be composed of two or more (e.g., 2-8, 3-6, 6-8, or more) sequence-specific DNA binding domains (e.g., zinc finger domains) fused to an effector endonuclease domain (e.g., the FokI endonuclease) (Porteus et al., Nat. Biotechnol. 23, 967-973 (2005); Kim et al. (2007) Hybrid restriction enzymes: Zinc finger fusions to Fok I cleavage domain, Proceedings of the National Academy of Sciences of USA, 93:1156-1160; U.S. Pat. No. 6,824,978; PCT Publication Nos. WO1995/09233 and WO1994018313).

The sequence-specific endonuclease of the methods and compositions described herein can be engineered, chimeric, or isolated from an organism. Endonucleases can be engineered to recognize a specific DNA sequence, by, e.g., mutagenesis (Seligman et al. (2002) Mutations altering the cleavage specificity of a homing endonuclease, Nucleic Acids Research 30: 3870-3879). Combinatorial assembly is a method where protein subunits form different enzymes can be associated or fused (Arnould et al. (2006) Engineering of large numbers of highly specific homing endonucleases that induce recombination to novel DNA targets, Journal of Molecular Biology 355: 443-458). These two approaches, mutagenesis and combinatorial assembly, may be combined to produce an engineered endonuclease with desired DNA recognition sequence.

The sequence-specific nuclease can be in the form of a protein or in the form of a nucleic acid encoding the sequence-specific nuclease, such as an mRNA or a cDNA.

### CRISPR Systems

The CRISPR (Clustered Regularly interspaced Short Palindromic Repeats) system exploits RNA-guided DNA-binding and sequence-specific cleavage of target DNA. A guide RNA (gRNA) are complementary to a target DNA sequence. The guide RNA/Cas combination confers site specificity to the nuclease. A single guide RNA (sgRNA) contains about 20 nucleotides that are complementary to a target genomic DNA sequence and a constant RNA scaffold region. The Cas (CRISPR-associated) protein binds to the guide RNA (gRNA) or sgRNA and the target DNA to which the gRNA or sgRNA binds and introduces a double-strand break. Geurts et al., Science 325:433 (2009); Mashimo et al., PLoS ONE 5:e8870 (2010); Carbery et al., Genetics 186:451-459 (2010); Tesson et al., Nat. Biotech. 29:695-696 (2011). Wiedenheft et al. Nature 482:331-338 (2012); Jinek et al. Science 337:816-821 (2012); Mali et al. Science 339:823-826 (2013); Cong et al. Science 339:819-823 (2013).

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

Cleavage of a gene region may comprise cleaving one or two strands at the location of the target sequence by the Cas enzyme. In one embodiment, such, cleavage can result in decreased transcription of a target gene. In another embodiment, the cleavage can further comprise repairing the cleaved target polynucleotide by homologous recombination with an exogenous template or donor DNA, wherein the repair results in an insertion, deletion, or substitution of one or more nucleotides of the target polynucleotide.

The terms “gRNA,” “guide RNA” and “CRISPR guide sequence” may be used interchangeably throughout and refer to a nucleic acid comprising a sequence that determines the specificity of a Cas DNA binding protein of a CRISPR/Cas system. A gRNA hybridizes to (complementary to, partially or completely) a target nucleic acid sequence in the genome of a host cell. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length.

As used herein, a “scaffold sequence,” also referred to as a tracrRNA, refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein. Exemplary scaffold sequences will be evident to one of skill in the art and can be found, for example, in Jinek, et al. Science (2012) 337(6096):816-821, Ran, et al. Nature Protocols (2013) 8:2281-2308, PCT Application No. WO2014/093694, and PCT Application No. WO2013/176772.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a gRNA sequence with a target polynucleotide sequence).

A gRNA can have a length ranging from about 12 nucleotides to about 100 nucleotides. For example, gRNA can have a length ranging from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the first segment (e.g., crRNA) can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. A gRNA can have fewer than 12 nucleotides or greater than 100 nucleotides.

sgRNA(s) can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

The present engineered exosome or extracellular vesicle may contain one, two or more sgRNAs targeting the DNA encoding one or more proteins or polypeptides.

In one embodiment, Cas protein may be a functional derivative of a naturally occurring Cas protein. In certain embodiments, the Cas enzyme comprises one or more mutations.

The Cas enzyme may be a type II, type I, type III, type IV or type V CRISPR system enzyme. In some embodiments, the Cas enzyme is a Cas9 enzyme (also known as Csn1 and Csx12).

Cas9 may be wild-type or mutant. In some embodiments, the endonuclease is a Cas9 homolog or ortholog. Cas9 may be any variant disclosed in U.S. Patent Publication No. 2014/0068797. Cas9 may be Type II-A, Type II-B, or Type II-C.

In some embodiments, the Cas9 is a modified form or a variant of the wild-type Cas9. In some instances, the modified form of the Cas9 protein comprises an amino acid change (e.g., deletion, insertion, or substitution) that reduces the naturally occurring nuclease activity of the Cas9 protein. In certain embodiments, the modified form of the Cas9 protein has less than 50%, less than 40%, less than 30%, less than 20%, less than 10%, less than 5%, or less than 1% of the nuclease activity of the corresponding wild-type Cas9 protein. In some cases, the modified form of the Cas9 protein has no substantial nuclease activity.

Cas9 may be from various species. Non-limiting examples of the Cas9 enzyme include Cas9 derived from Streptococcus pyogenes (S. pyogenes), S. pneumoniae, Staphylococcus aureus, Neisseria meningitidis, Streptococcus thermophilus (S. thermophilus), or Treponema denticola. The Cas enzyme may also be derived from Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

In certain embodiments, the Cas enzyme is Cas9, Cpf1, C2c1, C2c2, C2c3, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof. In one embodiment, the Cas enzyme is Cas9.

Cas9 harbors two independent nuclease domains homologous to HNH and RuvC endonucleases, and by mutating either of the two domains, the Cas9 protein can be converted to a nickase that introduces single-strand breaks (Cong, et al. Science 339:819-823 (2013)). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism.

In some embodiments, the nucleotide sequence encoding the Cas9 endonuclease is further modified to alter the activity of the protein. In some embodiments, the Cas9 endonuclease is a catalytically inactive Cas9. For example, dCas9 contains mutations of catalytically active residues (D10 and H840) and does not have nuclease activity. Alternatively or in addition, the Cas9 endonuclease may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g. CRISPR interference (CRISPRi)). In some embodiments, dCas9 is fused to an activator domain, such as VP64 or VPR. In some embodiments, such dCas9 fusion proteins are used with the constructs described herein for gene activation (e.g., CRISPR activation (CRISPRa)). In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation. In some embodiments, dCas9 or Cas9 is fused to a Fok1 nuclease domain. In some embodiments, Cas9 or dCas9 fused to a Fok1 nuclease domain is used for genome editing. In some embodiments, Cas9 or dCas9 is fused to a fluorescent protein (e.g., GFP, RFP, mCherry, etc.). In some embodiments, Cas9/dCas9 proteins fused to fluorescent proteins are used for labeling and/or visualization of genomic loci or identifying cells expressing the Cas endonuclease.

Differential gene expression can be achieved by modifying the efficiency of gRNA base-pairing to the target sequence (Larson et al., 2013, CRISPR interference (CRISPRi) for sequence-specific control of gene expression, Nature Protocols 8 (11): 2180-96). Modulating this efficiency may be used to create an allelic series for any given gene, creating a collection of hypomorphs and hypermorphs. These collections can be used to probe any genetic investigation. For hypomorphs, this allows the incremental reduction of gene function as opposed to the binary nature of gene knockouts.

CRISPR interference (CRISPRi) or CRISPR activation (CRISPRa) may be used in the present systems and methods.

CRISPRi is a transcriptional interference technique that allows for sequence-specific repression of gene expression and/or epigenetic modifications in cells (Qi et al., (2013) Repurposing CRISPR as an RNA-guided platform for sequence-specific control of gene expression. Cell 152 (5):1173-83). CRISPRi regulates gene expression primarily on the transcriptional level. CRISPRi can sterically repress transcription, e.g., by blocking transcriptional initiation or elongation. The target sequence may be the promoter and/or exonic sequences (such as the non-template strand and/or the template strand), and/or introns (Ji et al., (2014). Specific gene repression by CRISPRi system transferred through bacterial conjugation. ACS Synthetic Biology 3 (12): 929-31). CRISPRi can also repress transcription via an effector domain. Fusing a repressor domain to a catalytically inactive Cas enzyme, e.g., dead Cas9 (dCas9), may further repress transcription. For example, the Krüppel associated box (KRAB) domain can be fused to dCas9 to repress transcription of the target gene (Gilbert et al., 2013, CRISPR-mediated modular RNA-guided regulation of transcription in eukaryotes. Cell 154 (2): 442-51).

CRISPRa utilizes the CRISPR technique to allow for sequence-specific activation of gene expression and/or epigenetic modifications in cells (Qi et al., (2013) Repurposing CRISPR as an RNA-guided platform for sequence-specific control of gene expression, Cell 152 (5):1173-83; Gilbert et al., (2013) CRISPR-mediated modular RNA-guided regulation of transcription in eukaryotes, Cell 154 (2):442-51). For example, a catalytically inactive Cas enzyme, e.g., dCas9, may be used to activate genes when fused to transcription activating factors. These factors include, but are not limited to, subunits of RNA Polymerase II and traditional transcription factors, such as VP16, VP64, VPR etc. Gilbert et al., 2014, Genome-Scale CRISPR-Mediated Control of Gene Repression and Activation, Cell 159 (3): 647-61.

The Cas endonuclease may be a Cpf1 nuclease. In some embodiments, the host cell expresses a Cpf1 nuclease derived from Provetella spp. or Francisella spp.

Prime editing CRISPR may also be used in the present systems and methods. See Anzalone et al. (2019) Search-and-replace genome editing without double-strand breaks or donor DNA, Nature 576:149-157. The guide RNA used in prime editing CRISPR, called prime editing guide RNA (pegRNA), is substantially larger than standard sgRNAs commonly used for CRISPR gene editing (>100 nt vs. 20 nt). The pegRNA is a sgRNA with a primer binding sequence (PBS) and the template containing the desired RNA sequence added at the 3′ end. Together, they form the PE:pegRNA complex, which is used to mediate genome editing within the cell.

First, an engineered prime editing guide RNA (pegRNA) that both specifies the target site and contains the desired edits) engages the prime editor protein. This primer editor protein consists of a Cas9 nickase fused to a reverse transcriptase. The Cas9 nickase part of the protein is guided to the DNA target site by the pegRNA. After nicking by Cas9, the reverse transcriptase domain uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Lastly, the editor guides resolution of the heteroduplex to favor copying the edit onto the unedited strand, completing the process.

The Cas9 nickase can be fused to the M-MLV reverse transcriptase (RT) including mutated versions to create the prime editor (PE) (PE1 and PE2).

Once the prime editor incorporates the edit into one strand, there is a mismatch between the original sequence on one strand and the edited sequence on the other strand. To guide heteroduplex resolution to favor the edit, the non-edited strand is nicked causing the cell to remake that strand using the edited strand as the template.

A third prime editing system called PE3 does this by including an additional sgRNA. Using this sgRNA, the prime editor nicks the unedited strand away from the initial nick site (to avoid creating a double strand break.), increasing editing efficiencies 2 to 3 fold with indel frequencies between 1-10%.

The other important component of prime editing is the prime editing guide RNA (pegRNA). The pegRNA is a guide RNA that also encodes the RT template, which includes the desired edit and homology to the genomic DNA locus. Sequence complementary to the nicked genomic DNA strand serves as a primer binding site (PBS). This PBS sequence hybridizes to the target site and serves as the point of initiation for reverse transcription. To optimize pegRNAs extending the pegRNA primer binding site to at least eight nucleotides enables more efficient prime editing. pegRNA may be between may be between 50-500 nucleotides in length.

### Inhibitory Nucleic Acids

In certain embodiments, the cargo or payload may be an inhibitory nucleic acid or polynucleotide that reduces expression of a target gene. Thus, the polynucleotide specifically targets a nucleotide sequence encoding a target protein or polypeptide.

The nucleic acid target of the polynucleotides may be any location within the gene or transcript of the target protein or polypeptide.

The inhibitory nucleic acids may be RNA interference or RNAi, an antisense RNA, a ribozyme, or combinations thereof.

“RNA interference”, or “RNAi” is a form of post-transcriptional gene silencing (“PTGS”), and comprises the introduction of, e.g., double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. RNAi can work in human cells if the RNA strands are provided as pre-sized duplexes of about 19 nucleotide pairs, and RNAi worked particularly well with small unpaired 3′ extensions on the end of each strand (Elbashir et al. Nature 411:494-498 (2001)).

RNAi may be small interfering RNA or siRNAs, a small hairpin RNA or shRNAs, microRNA or miRNAs, a double-stranded RNA (dsRNA), etc.

The cargo or payload may be a short RNA molecule, such as a short interfering RNA (siRNA), a small temporal RNA (stRNA), and a micro-RNA (miRNA). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

Alternatively, a polynucleotide encoding an siRNA or shRNA may be used.

The inhibitory nucleic acids may be an antisense nucleic acid sequence that is complementary to a target region within the mRNA of a target protein or polypeptide. The antisense polynucleotide may bind to the target region and inhibit translation. The antisense oligonucleotide may be DNA or RNA or comprise synthetic analogs of ribo-deoxynucleotides.

An antisense oligonucleotide can be, for example, about 7, 10, 15, 20, 25, 30, 35, 40, 45, 50, 55, 60, 65, 70, 75, 80, or more nucleotides in length.

The cargo or payload may be a ribozyme. Ribozymes can be chemically synthesized and structurally modified to increase their stability and catalytic activity using methods known in the art.

### Antibodies

The cargo or payload may be an antibody or a fragment (e.g., an antigen-binding portion) thereof.

The antibody or antigen-binding portion thereof may be the following: (a) a whole immunoglobulin molecule; (b) a single-chain variable fragment (scFv); (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human/humanized antibodies.

### Donor/Template Polynucleotides

The cargo or payload may also contain a donor/template polynucleotide (e.g., DNA). The donor/template polynucleotide may be a single-stranded donor DNA or a double-stranded donor DNA.

A DNA double-strand break at a defined site in the genome (e.g., produced by a Cas-family nuclease) may or may not be repaired by the DNA repair machinery. The most involved DNA repair pathways are non-homologous end joining (NHEJ) and homology directed repair (HDR). NHEJ usually leads to a small insertion or deletion and thus gene knockout. NHEJ can be exploited for gene knockout by the introduction of a premature STOP-codon or frame shift of genetic reading. HDR with a donor/template polynucleotide can lead to insertion of a desired sequence of DNA. Gene correction, epigenetic modulation, and knock-in can be obtained through HDR, by addition of a donor/template DNA to the machinery which leads to repair complementary to the provided template.

Depending on the desired genetic manipulation, various components of CRISPR/Cas may be delivered as a cargo or payload by the present engineered exosome or extracellular vesicle: (a) a minimal Cas/gRNA pair for gene disruption/mutation, (b) Cas/gRNA and a donor/template DNA for gene correction, (c) Cas/gRNA and a desired gene for gene insertion (e.g., a donor/template DNA), or (d) Cas9 and two gRNAs for the complete deletion of a gene (or a portion of a gene).

In one embodiment, the donor/template polynucleotide may result in the expression of a corrected gene, which can restore or correct the function of the disease-related gene or fragment after the deletion/mutation/truncation of endogenous gene(s) or fragments.

In one embodiment, the cargo or payload comprise a gRNA and a single-stranded donor/template DNA. The gRNA is designed to be within 10 nucleotides of HDR. The 5′ and 3′ homology arms are about 30-40 nucleotides. Each end of the donor/template DNA may contain two phosphonothioates to increase HDR efficacy.

In certain embodiments, the cargo or payload may also include a desired gene for gene insertion.

In one embodiment, the desired gene for gene insertion is a codon-modified polynucleotide for a gene of interest. In one embodiment, the donor/template polynucleotide is codon modified to be unrecognizable by the DNA digesting agent (e.g., gRNA or sgRNA). Such donor sequence may encode at least a functional fragment of the protein lacking or deficient in the cell.

Alternatively, if the present composition and method deletes, destroys, or truncates only the mutated form of a gene or a fragment (e.g., a mutant allele), and leave the wild type form (e.g., a wildtype allele) intact, donor template or wild type gene sequence that is supplemented to the cells or a patient may not be codon-modified. Under such circumstances, the DNA digesting agent (e.g., gRNA or sgRNA in combination with a Cas enzyme) would be designed to recognize and target only the mutated form of a disease-related gene (and not recognize and target a wild type form of the gene).

The donor/template polynucleotide may be integrated into the endogenous gene. Such targeted integration may be accomplished by homologous recombination.

In one embodiment, the donor/template polynucleotide is flanked by an upstream and a downstream homology arm. The homology arms, which flank the donor sequence, correspond to regions within the targeted locus.

Alternatively, the donor/template polynucleotide (whether codon-modified or not) of a gene of interest or fragment is not integrated into the endogenous gene. The donor/template polynucleotide may offer expression without integration into the host genome.

### Diagnostic Agents

The cargo or payload may comprise a diagnostic agent including but not limited to radioactive tracers or radionuclides used for positron emission tomography (PET) and other scans, including but not limited to carbon-11, nitrogen-13, oxygen-15 and fluorine-18. These agents can be more precisely delivered to the tissue of interest by loading them on an engineered exosome which either artificially or naturally targets the tissue of interest, i.e., the tissue that is being scanned, thus, reducing off-target delivery of radioisotopes.

### Exosome or Extracellular Vesicle Loading

Exosomes or extracellular vesicles may be loaded with different cargoes by, e.g., transduction, expression in producing cells, electroporation, transfection, microinjection, etc. Cultured cells may be engineered to express various cargoes by, e.g., transduction, electroporation, transfection, microinjection, etc. Exosomes or extracellular vesicles are then produced from these cells which are loaded with the desired cargo(s). The payload may be introduced into the cell in the form of a DNA (e.g., cDNA), mRNA and protein.

Proteins, peptides, or polypeptides may be loaded into an exosome or extracellular vesicle by transfection or electroporation. Proteins or polypeptides may also be introduced into a cell from which an engineered exosome or extracellular vesicle may be generated.

The nucleic acids may be delivered to cultured cells in vitro. Nucleic acids can be delivered as part of a larger construct, such as a plasmid or viral vector, or directly, e.g., by electroporation, lipid vesicles, viral transporters, microinjection, heat shock, and biolistics. Non-limiting examples of methods to introduce nucleic acids into cells include lipofectamine transfection, calcium phosphate co-precipitation, electroporation, DEAE-dextran treatment, microinjection, lipid-mediated transfection, viral infection, chemical transformation, electroporation, lipid vesicles, viral transporters, ballistic transformation, pressure induced transformation, viral transduction, particle bombardment, and other methods known in the art.

In some embodiments, a population of cells are transiently or non-transiently (e.g., stably) transfected or infected with one or more vectors described herein. In some embodiments, a cell infected or transfected with one or more vectors described herein is used to establish a cell line comprising one or more sequences encoding one or more of the present CRISPR components.

Suitable cells include, but are not limited to, mammalian cells (e.g., human cells, mouse cells, rat cells, etc.), primary cells, stem cells, immune cells, and any other type of cells known to those skilled in the art.

In certain embodiments, the engineered exosome or extracellular vesicle may be loaded with (i) a DNA polynucleotide encoding a Cas enzyme or a variant thereof, (ii) a RNA polynucleotide encoding a Cas enzyme or a variant thereof, or (iii) a Cas enzyme or a variant thereof.

The cells may express a Cas enzyme (e.g., Cas9 expressing cells). For example, the cells may be stably transfected with DNA encoding Cas9. The cells may have DNA encoding Cas9 stably integrated. Expressing the nucleic acid molecule may also be accomplished by integrating the nucleic acid molecule into the genome. In certain embodiments, cultured cells can be engineered to express, either through stable expression or transient expression, to express a Cas enzyme (e.g., CAS9). Exosomes or extracellular vesicles can be generated from these cells which also express a Cas enzyme (e.g., CAS9). A gRNA or sgRNA is then introduced (e.g., by transfection or electroporation) into the exosomes or extracellular vesicles which now contain both a Cas enzyme and a gRNA/sgRNA.

In some embodiments, the Cas endonuclease and the nucleic acid encoding the gRNA are provided on the same nucleic acid (e.g., a vector). In some embodiments, the Cas endonuclease and the nucleic acid encoding the gRNA are provided on different nucleic acids (e.g., different vectors). Alternatively or in addition, the Cas endonuclease may be loaded into the exosome or extracellular vesicle in protein form.

There are several formats in which the gRNA and Cas proteins can be delivered to the cell that will be used to produce exosomes or extracellular vesicles: plasmids or viral vectors that carry Cas9 and gRNA genes in gene-based delivery; Cas mRNA and a gRNA in RNA-based delivery; or Cas protein and a synthetic gRNA in protein-based delivery. A same vector or different vectors may be used to encode the CAS protein and sgRNA. In some embodiments, a Cas enzyme in combination with (and optionally complexed with) a gRNA or a sgRNA, is delivered to a cell. The purified Cas9 protein is positively charged and can efficiently form a complex with sgRNA, which is called Cas9/sgRNA ribonucleoprotein complexes (RNPs). Therefore, the CAS9 protein complexed with sgRNA can be directly delivered. The RNPs may be introduced to exosomes or extracellular vesicles by electroporation, transfection, or microinjection.

### Targeting Moieties and Therapeutic Molecules

The targeting moiety may be a cell-specific, tissue-specific, or organ-specific targeting moiety. The targeting moiety may be an integrin, a cell-specific, tissue-specific, or organ-specific antibody, a cell-specific, tissue-specific, or organ-specific receptor, or a cell-specific, tissue-specific, or organ-specific polypeptide/peptide. The engineered exosome or extracellular vesicle will be targeted to and taken up selectively by the desired cells/tissue/organ. In some embodiments, the targeting moiety targets a cell, tissue or organ including but not limited to the lung, liver and brain.

The targeting moiety may be an integrin, such as the αvβ5 integrin, the α6β4 integrin, and the α6β1 integrin, etc. For example, exosomes expressing the αvβ5 integrin may specifically bind to Kupffer cells, thus delivering the engineered exosome or extracellular vesicle specifically to the liver. The α6β4 and α6β1 integrins may bind to lung fibroblasts and epithelial cells, directing the engineered exosome or extracellular vesicle specifically to the lung.

Low-density lipoprotein (LDL) receptor-related protein (LRP) or anti-EGFR antibody or cyclic arginine-glycine-aspartic acid (RGD) peptide, or αvβ3 integrin, or peptides targeted toward Nicotinic acetylcholine receptors (nAChRs), or peptides derived from rabies virus glycoprotein-RVG29, or transferrin or peptide and ligands for transferrin receptor (TfR), the low density lipoprotein receptor (LDLR), the insulin receptor, nicotinic acetylcholine receptors, or blood brain barrier specific peptides could be introduced on the surface of exosomes or extracellular vesicles for brain targeting.

The targeting moiety may be a chimeric protein with a structural protein/polypeptide (e.g., an integrin, antibody, peptide, etc.) fused with at least a portion of an exosomal marker such as CD63. The DNA encoding the chimeric protein may be in a suitable expression vector having the CD63 promoter. The chimeric protein can be expressed in cells. The exosomes produced from these cells can express the chimeric protein. The chimeric protein is multifunctional with the properties of both of the exosomal marker (such as CD63) which is a ubiquitous membrane protein, and a targeting moiety (e.g., an integrin, antibody, peptide, etc.). Extracellular vesicle or exosome markers include CD9, CD63, CD81, LAPM1, TSG101, and combinations thereof.

In one embodiment, the translational 3′-terminus of the CD63 is deleted, as is the promoter of the 5′-terminus of the second structural gene. The two genes will be then ligated in-frame with an intervening linker and will be expressed in cells. After transcription and translation, the exosomes produced from those cells will produce one single polypeptide chain with properties of both of the original gene products CD63 and targeting moieties (integrins, antibodies, etc.). This will lead to the formation of a multifunctional protein with the properties of both of the original gene products: CD63 which is a ubiquitous membrane protein and integrin fusion protein which leads to the specific function and targeting moieties in the exosomes to mediate organ/tissue specific delivery.

With specific delivery, expression of the target gene may not be substantially reduced in other cells/tissues/organs, i.e., cells/tissues/organs which are not desired target cells/tissues/organs. Thus, in such embodiments, the level of the target protein remains substantially the same or similar in non-target cells in the course of or following treatment.

The engineered exosome or extracellular vesicle may be administered to a subject locally or systemically. For local administration, the engineered exosome or extracellular vesicle may be injected directly into the desired target site, e.g., in a depot or sustained release formulation.

The therapeutic molecule may be an antibody, a small molecule, gRNA, mRNA, microRNA, micoRNA mimic, microRNA inhibitor, siRNA, synthetic small RNA, synthetic RNA, anti-sense, CRISPR/CAS, a gRNA, a pegRNA, CAS proteins, a peptide, a cytokine, a signaling molecule, or combinations thereof.

### Vectors

As used herein, a “vector” may be any of a number of nucleic acids into which a desired sequence or sequences may be inserted for transport between different genetic environments or for expression in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art.

Vectors include, but are not limited to, viral vectors, plasmids, cosmids, fosmids, phages, phage lambda, phagemids, and artificial chromosomes.

Viral vectors may be derived from DNA viruses or RNA viruses, which have either episomal or integrated genomes after delivery to the cell. See Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Viral vectors may be derived from retroviruses (including lentiviruses), replication defective retroviruses (including replication defective lentiviruses), adenoviruses, replication defective adenoviruses, adeno-associated viruses (AAV), herpes simplex viruses, and poxviruses. In some embodiments, the vector is a lentiviral vector. Options for gene delivery of viral constructs are known (see, e.g., Ausubel et al., Current Protocols in Molecular Biology, John Wiley & Sons, New York, 1989; Kay, et al., 2001 Nat. Medic. 7(1):33-40; and Walther W. and Stein U., 2000 Drugs, 60(2): 249-71).

Any subtype, serotype and pseudotype of lentiviruses, and both naturally occurring and recombinant forms, may be used as a vector for the present systems and methods. Lentiviral vectors may include, without limitation, primate lentiviruses, goat lentiviruses, sheep lentiviruses, horse lentiviruses, cat lentiviruses, and cattle lentiviruses.

The term AAV covers all subtypes, serotypes and pseudotypes, and both naturally occurring and recombinant forms. AAV viral vectors may be selected from among any AAV serotype, including, without limitation, AAV1, AAV2, AAV3, AAV4, AAV5, AAV6, AAV7, AAV8, AAV9, AAV10 or other known and unknown AAV serotypes. Pseudotyped AAV refers to an AAV that contains capsid proteins from one serotype and a viral genome of a second serotype.

A variety of vectors may be used to deliver CRISPR components to the targeted cells and/or a subject. In some embodiments, one or more sequences encoding one or more of the present CRISPR components are part of the same vector, or two or more vectors.

The constructs encoding the present CRISPR components can be delivered to the cell using one or more vectors (e.g., 1, 2, 3, 4, 5, 6, 7, 8, 9, or more vectors). One or more sequence encoding one or more CRISPR components can be packaged into a vector. A Cas enzyme can be packaged into the same, or alternatively separate, vectors.

Vectors may further contain one or more marker sequences suitable for use in the identification of cells which have or have not been infected, transformed, transduced or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein, red fluorescent protein). Molecular Cloning: A Laboratory Manual, Fourth Edition, Cold Spring Harbor Laboratory Press, 2012. Current Protocols in Molecular Biology, John Wiley & Sons, Inc.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system).

In some embodiments, a prokaryote is used to amplify copies of a vector and express one or more nucleic acids, such as to provide a source of one or more proteins for delivery to a host cell or host organism. In some embodiments, a vector is a yeast expression vector. In some embodiments, a vector drives protein expression in insect cells using baculovirus expression vectors.

In some embodiments, a vector is capable of driving expression of one or more sequences in mammalian cells using a mammalian expression vector. Non-limiting examples of promoters include those derived from polyoma, adenovirus 2, cytomegalovirus, simian virus 40, and others disclosed herein and known in the art. Sambrook, et al., Molecular Cloning: A Laboratory Manual, 4th ed., Cold Spring Harbor Laboratory, Cold Spring Harbor Laboratory Press, Cold Spring Harbor, N.Y., 2012.

### Pharmaceutical Compositions

The present disclosure provides a pharmaceutical composition comprising the present engineered exosomes or extracellular vesicles. The present disclosure provides uses of the present engineered exosomes or extracellular vesicles for manufacturing a medicament for use in treating a condition or disorder.

In some embodiments, the present engineered exosomes or extracellular vesicles may be mixed with a pharmaceutically acceptable carrier to form a pharmaceutical composition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

The present engineered exosomes or extracellular vesicles may be delivered to a cell by contacting the cell with the exosomes or extracellular vesicles.

The present engineered exosomes or extracellular vesicles or the present composition may be delivered/administered to a subject by any route, including, without limitation, intravenous, intracerebroventricular (ICV) injection, intracisternal injection or infusion, oral, transdermal, ocular, intraperitoneal, subcutaneous, implant, sublingual, subcutaneous, intramuscular, rectal, mucosal, ophthalmic, intrathecal, intra-articular, intra-arterial, sub-arachinoid, bronchial and lymphatic administration. The present composition may be administered parenterally or systemically. The present composition may be administered locally.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

### Conditions to be Treated

The present application provides methods for treating a disorder in a subject comprising administering to the subject the present composition. The present engineered exosomes or extracellular vesicles are administered in a therapeutically effective amount. The present engineered exosome or extracellular vesicle may be delivered/administered to a subject for treating a condition, disorder or disease. The present compositions and methods may modify or alter (e.g., increase or decrease) the expression of one or more genes.

The conditions to be treated by the present engineered exosome or extracellular vesicle include a variety of diseases including cancer, genetic diseases such as cystic fibrosis, and autoimmune diseases. The present compositions and methods may be used in regenerative medicine. The conditions to be treated by the present engineered exosome or extracellular vesicle include a lung disease such as lung cancer.

The present compositions and methods may be used to deliver a small molecule to treat cancer, fibrosis, an inflammatory disease, a genetic disease, etc.

The conditions to be treated by the present engineered exosome or extracellular vesicle include, without limitation, a hematologic malignancy, lung cancer, ear, nose and throat cancer, colon cancer, melanoma, pancreatic cancer, mammary cancer, prostate cancer, breast cancer, ovarian cancer, basal cell carcinoma, biliary tract cancer; bladder cancer; bone cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

The present compositions and methods may be used to treat liver fibrosis, inflammatory conditions of the lung including those caused by viruses such as SARS-CoV-2, bronchitis and sepsis.

The present compositions and methods may be used to correct a genetic disease, to correct a cancer-related mutation, or to engineer T cells, macrophages, natural killer cells or dendritic cells for immunotherapy (e.g., cancer immunotherapy).

Cystic fibrosis (CS) is a life-limiting autosomal recessive disorder caused by disruption of transmembrane conductance regulator (CFTR) protein. CFTR functions as a chloride channel regulated by cyclic AMP (cAMP)-dependent phosphorylation. Loss of function mutation in CFTR leads to abnormally viscous secretions in the airways of the lungs and in the ducts of the pancreas in individuals with cystic fibrosis which cause obstructions that lead to inflammation, tissue damage and destruction of both organ systems. The present compositions and methods may be used to edit/correct this mutation. In one embodiment, the gRNA targets a sequence corresponding to codon 489 of a CFTR protein.

The present compositions and methods may be used to treat lung cancer such as non-small cell lung cancer (NSCLC) with KRAS mutation. KRAS is one of the most commonly altered oncogenes acting as tumor genomic drivers in NSCLC. The present compositions and methods may be used to edit/correct KRAS mutation.

The present compositions and methods may be used for targeted editing of cancer cells. In one embodiment, the payload may contain dead CAS9 (dCAS) for transcriptional activation/repression and epigenetic control of cancer cells. For example, multiple cancer suppressor genes can be upregulated using this method in cancer cells or supporting tumor microenvironment. The current system is programmable to be used for genome editing with other Cas proteins such as Cas12a for multiplex genome engineering.

The present compositions and methods may be used for various genome-editing applications, such as multiplex genome engineering, epigenetic modification, RNA targeting, activation or repression of target genes using CRISPR.

### Kits

The present disclosure also encompasses kits containing the present engineered exosomes or extracellular vesicles.

In some embodiments, the kit comprises the present engineered exosomes or extracellular vesicles and instructions for using the kit. Elements may be provided individually or in combinations.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form).

### EXAMPLES

This invention will be better understood from the Experimental Details, which follow. However, one skilled in the art will readily appreciate that the specific methods and results discussed are merely illustrative of the invention as described more fully in the claims that follow thereafter.

### Example 1—Materials and Methods for Examples 2-8 Exosome Isolation

Cell culture supernatants were centrifuged at 1500 g for 10 minutes to remove cells and 10000×g for 25 minutes to deplete residual cellular debris. Supernatant were serially filtered with 0.8 μm, 0.44 μm and 0.2 μm filters. The filtered supernatant was used to precipitate exosomes with Exoquick-TC or supernatants were condensed using Amicon Ultra-15 Centrifugal Filter Devices or exosomes were participated using ultracentrifugation. Ultracentrifugation was performed at 100,000 g for 90 minutes using fixed angle 75 Ti rotor at 4° C. Positive selection of exosomes using surface markers such as CD63 were done by using CD63-anti-immunomagnetic capturing method with a primary anti-CD63 followed by corresponding secondary antibody coupled to magnetic beads and were separated using magnetic column isolation devices. After isolation, exosomes were re-suspended in Dulbecco's phosphate-buffered saline.

### Exosome Loading

Exosomes were loaded with different cargoes using an optimized protocol of electroporation or using transfection reagents. Briefly, different gRNAs, single-stranded DNA (ssDNAs), double-stranded DNA (dsDNA), microRNA-mimic, small synthetic RNA, microRNA inhibitor, siRNA or control siRNA, or control microRNA mimics and inhibitors were loaded into the exosomes with electroporation or transfection reagents. For electroporation, isolated exosomes in Dulbecco's phosphate-buffered saline were diluted in electroporation buffer in 1:1 ratio.

The mixture of nucleic acid and exosomes were transferred into cold 0.2 cm electroporation cuvettes and electroporated at 150 kV-200 kV and 100 μF. The exosomes were treated with one unit of RNase od DNase to eliminate free-floating nucleic acids and exosomes and re-isolated using any of the methods described in the isolation section. For loading of any of the cargoes with transfection reagents, the complex of cargo and transfection reagent was formed and added to the exosomes and exosomes were re-isolated as described in the isolation section.

### Immunoblotting

Whole-cell lysates or exosome lysate were prepared on ice using RIPA lysis buffer solution, 1% protease, and phosphatase inhibitor cocktail. Protein concentrations were determined with a Broadford Protein Assay. Lysate protein was subjected to 10% SDS-PAGE and transferred to a nitrocellulose membrane, After blotting, membranes were probed with primary antibody at 4° C. overnight. The following primary Abs were used: anti-CAS9, anti-CD63, anti-beta4. After washing three times with TBST, membranes were incubated with HRP-conjugated secondary goat anti-rabbit or anti-mouse antibody for 2 h at room temperature. Blots were developed with the clarity max western ECL detection system according to the manufacturer's instructions and images were captured from iBrightCL1000.

siRNA Transfection

THP1 cells, primary human monocytes/macrophages, or HCC827 were cultured in 12 well plates at 500,000 cells/well for 24 h in DMEM or RPMI complete culture medium before the experiment. Transfection complex was prepared by incubating diluted siRNA and Lipfectamine RNAiMax in 100 μl of Opti-MEM for 5 minutes at room temperature prior to being added to each well. On the day of transfection, cells were washed once with sterile phosphate-buffered saline (PBS), and the Opti-MEM culture medium was added. Cells were transfected with different siRNAs or negative control siRNA.

### RNA Isolation and Quantification

Cell and exosomes were lysed in Qiazol reagent and total RNA was isolated using a Direct-zol RNA MiniPrep isolation kit (Zymo Research Corp). The extracted RNA was eluted with 25-35 μL of RNase-free water. The quantity and quality of the RNA were determined by NanoDrop 1000 (260/280 and 260/230 ratios) or Agilent Bioanalyzer 2100 with a Small RNA Chip for exosomal RNA.

microRNA Analysis

TaqMan microRNA Assays was used for the detection of microRNA-34a and Cel-39-3p. Reverse transcription (30 min, 16° C.; 30 min, 42° C.; 5 min 85° C.) was done using a TaqMan stem loop primer, 10 ng RNA input, TaqMan primers and a microRNA reverse transcription kit. Quantitative real-time PCR was perfumed in Bio-Rad CFX96 iCycler using TaqMan Universal PCR Master Mix. RNU-48 was used as a internal control to normalize the Ct values between the samples. TaqMan Pri-microRNA Assay was done using FAM dye-labeled TaqMan with GAPDFI as an internal control. microRNA levels were normalized and the relative expression levels of specific microRNA were presented by 2−ΔΔCt.

### Injection of Loaded Exosomes to Mice

Prior to injecting into recipient mice, microRNA loaded exosomes or gRNA loaded exosomes or single-strand DNA loaded exosomes, or scramble loaded exosomes or fluorescently labeled exosomes or synthetic Cel-39 loaded exosomes resuspended in BPS were brought to room temperature and vortexed and injected. 100-150 μl exosomes were injected into recipient mice (intravenous [i.v.]). Blood was drawn and animals were perfused using our standardized laboratory protocol. To rule out any blood contamination, red blood lysis buffer was used as per manufacturer's instructions.

### Mismatch Assay (T7 Endonuclease Assay)

Genomic DNA was extracted from exosome treated cells or other control conditions using the Zymo genomic DNA purification kit. 200 ng of genomic DNA was used for PCR amplification. Amplified PCR products were diluted by a factor 2 and complemented with Buffer 2 (New England Biolabs) to a final concentration of 1×. The PCR amplicons were then heat denatured at 95° C. followed by a cool down to 20° C. with a 0.1° C./s ramping. Heteroduplexes were incubated for 30 minutes at 37° C. in the presence of 10 units of T7 Endonuclease I. Specimens were run on a 2.5% agarose gel to assess editing efficiency.

### CRISPR Amplicon Sequencing

High-throughput CRISPR next-generation sequencing was performed to detect the frequency of mutations. Target-specific PCR primer binding site was designed to produce 200-280 bp, and the sequence was completely covered by sequencing. The TIDE algorithm was used to quantify the genome-editing efficacy.

### Production of Stable Mutated Cell Lines

Dorsha, ALX, and HNRPA1B were targeted using specific Cas-gRNA complex in the form of ribonucleoprotein (RNP) using fluorescently labeled CRISPR-Cas9 tracrRNA. At least two guide RNAs were designed against each protein. Single-cell derived knockout clones which were successfully internalized CRISPR-Cas9 tracrRNA, were selected by fluorescence-activated cell sorting in 96 well plates. Individual single-cell derived knockout clones were expanded knock out of proteins was confirmed by Western blot, and new knockout cell lines were established harboring those mutations.

### Fluorescent Microscopy and Flow Cytometry

Exosomes were labeled with PKH26 red fluorescent cell linker kit or di-8-ANEPPS according to the manufacturers' instruction. Labeled exosomes were re-isolated and co-cultured with human primary macrophages and then washed off. The uptake of labeled exosomes by recipient cells was visualized by a Zoe Fluorescent imager (Bio-Rad). Labeled exosome uptake was also quantified using a BD Fortessa flow cytometer.

### Directed Homology Repair (HDR) Using Engineered Exosomes

Engineered exosomes derived from Thp1 cell line or Thp1 cell line expressing CAS9 (CAsexo) were used for the cargo transfection. CASexo was loaded with ssDNA donor template containing Flag tag and gRNA against DDX3 to include the Flag at AUG codon. Different concentration of ssDNA donor template was used 0.5 μM, 1 μM and 3 μM. Further, exosomes were either transfected with DCLREIC siRNA and XRCC5 siRNA. After each transfection the exosomes were reprecipitated using ExoQuick-TC for purification according to the manufacturer's Instruction and stored at −80° C. further use. The insertion of Flag tag was checked through PCR.

### Statistical Analysis

Based on data distribution, one-way analyses of variance (ANOVA) or Kruskal-Wallis nonparametric test were used to compare different groups. Student's t test or Mann-Whitney U test were performed for comparing two groups. Data is presented as mean/standard error of mean. P values less than 0.05 was considered as statistically significant.

### Example 2—Production of Exosomes Devoid of Endogenous Nucleic Acids

To make exosomes biologically safe and eliminate the unwanted side effects associated with endogenous exosomal cargo, exosomes have been engineered to contain minimal endogenous nucleic acids in order to minimize unwanted biological effects associated with endogenous exosomal RNA. Exosomes can then be loaded with desired cargos or payloads including but not limited to nucleic acids (e.g., sgRNA, template DNA, siRNA, small molecules, and miRNA inhibitor/mimic or siRNA).

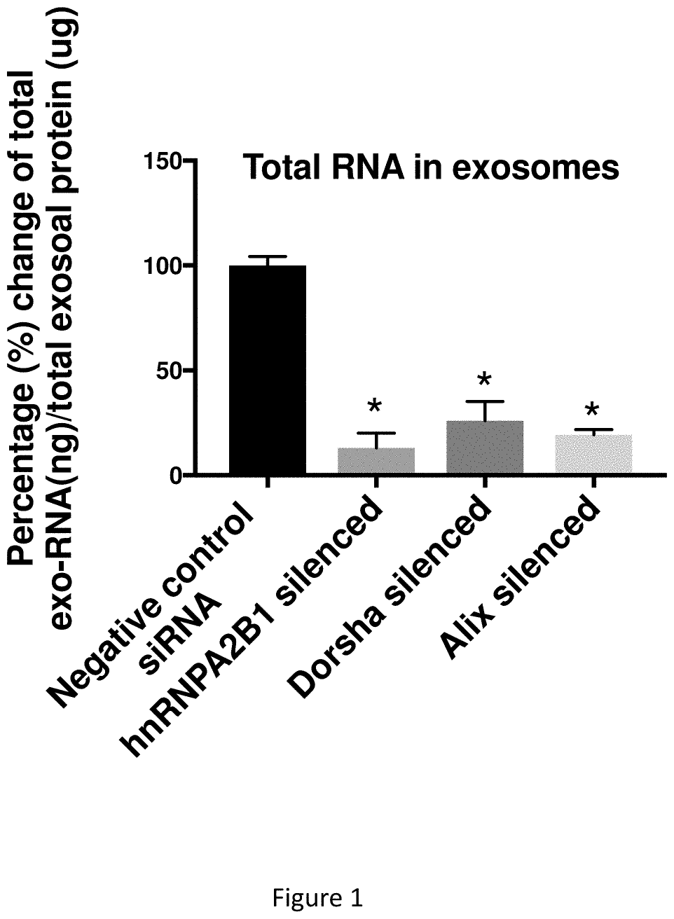

To make the exosomes devoid of any endogenous RNAs, heterogeneous nuclear ribonucleoprotein A2B1 (hnRNPA2B1) were silenced using Dorsha processing and Alix processing as described in Example 1. Exosomes were generated with minimal endogenous RNAs (FIG. 1). The protein heterogeneous nuclear ribonucleoprotein A2B1 (hnRNPA2B1) specifically binds exosomal RNA through the recognition of specific motifs, controlling their loading into exosomes (Villarroya-Beltri et al. 2013). Thus, by knocking down hnRNPA2B1, the key player in sorting RNAs into exosomes, engineered exosomes were generated which were almost free of any endogenous RNAs, making them ideal vehicles for delivery of nucleic acids (e.g., sgRNA, single-stranded DNA) as they will induce minimal off-target effects. Dorsha functions as the initiator of microRNA biogenesis by cleaving pri-miRNA to mature form of microRNA and ALIX mediate nucleic acid loading into the exosomes (Han et al. 2004; lavello et al 2016). Thus, their silencing leads to presence of minimal endogenous nucleic acid loading in the exosomes.

### Example 3—Introducing Targeting Moieties into the Exosomes

To further optimize the system, exosomes have been further engineered to express tissue specific targeting moieties for organ- and cell-specific delivery. Particularly, exosomal integrins were found to initiate organ colonization by fusing with target cells of specific tissues, leading to metastatic niche formation (Shimaoka et al. 2019; Hoshino et al. 2015).

Exosomes expressing the αvβ5 integrin were found to specifically bind to Kupffer cells, thus identifying the liver as the target organ for pre-metastatic niche formation (Hoshino et al. 2015). Similarly, exosomal α6β4 and α6β1 integrins were found to bind to lung fibroblasts and epithelial cells, directing exosomes to the lung (Hoshino et al. 2015). Using these specific tissue moieties for natural exosomes, exosomes were engineered to express chimeric proteins by recombinant means by fusing structural genes (e.g., integrins, antibodies, peptides) of the proteins in a suitable expression vectors expressed with the promotor of exosomal CD63 (a ubiquitous marker for exosomes).

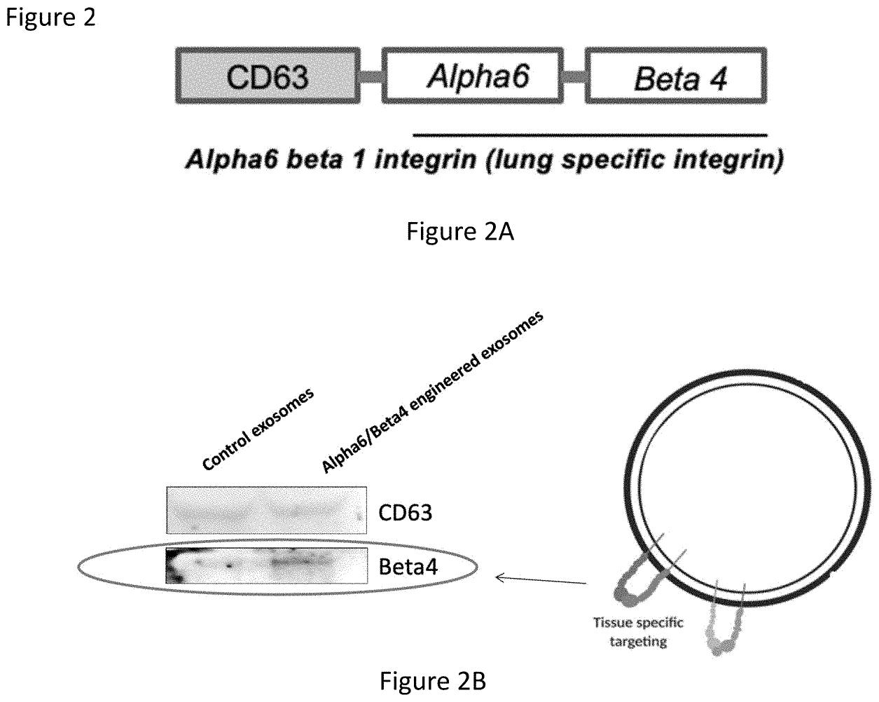

In this example, integrin α6β4 was used (FIG. 2A). The genes were then ligated in-frame with an intervening linker and expressed in cells. After transcription and translation, the exosomes produced from those cells produced one single polypeptide chain and expressed chimeric protein (FIG. 2B).

This approach led to the formation of a multifunctional protein with the properties of both of the original gene products: CD63, which is a ubiquitous membrane protein and a lung specific targeting moiety, alpha 6 beta 4 (α6β4) integrin.

### Example 4—Engineered Exosomes Loaded with Small Interfering RNAs, Small Molecules, and microRNA Mimics and Inhibitors

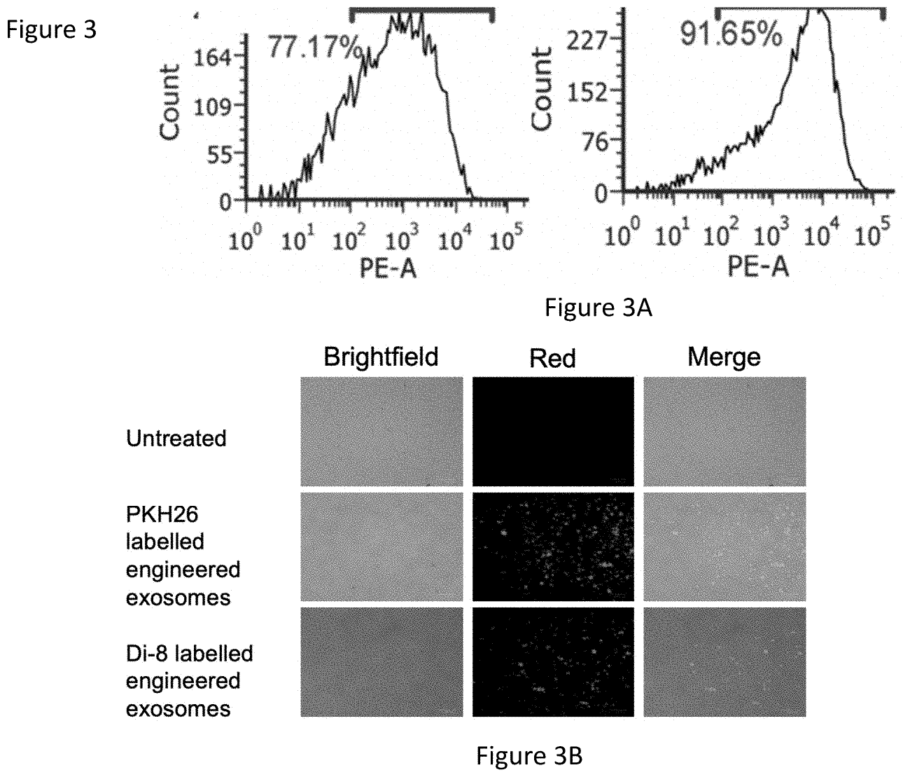

To show that the engineered exosomes are taken up by cells, the engineered exosomes from Example 2 were fluorescently labeled with PKH26 red fluorescent dye or small molecule dye di-8-ANEPPS and co-cultured with human macrophages as described in Example 1. di-8-ANEPPS are small molecules that fluoresce in response to electrical potential change in their environment. There was a dose depended uptake of fluorescently labeled exosomes after 1 hour of co-culture as depicted by flow cytometry (FIG. 3A) and fluorescent microscope (FIG. 3B). Coculture of engineered exosomes labeled with small molecule di-8-ANEPPS resulted in active internalization of exosomes by the recipient cells (FIG. 3B).

The engineered exosomes were then loaded with a synthetic version of either the C. elegans microRNA, cel-miR-39-3p, microRNA-34a mimic, and microRNA-34a inhibitor, and co-cultured with HCC827 cells as described in Example 1.

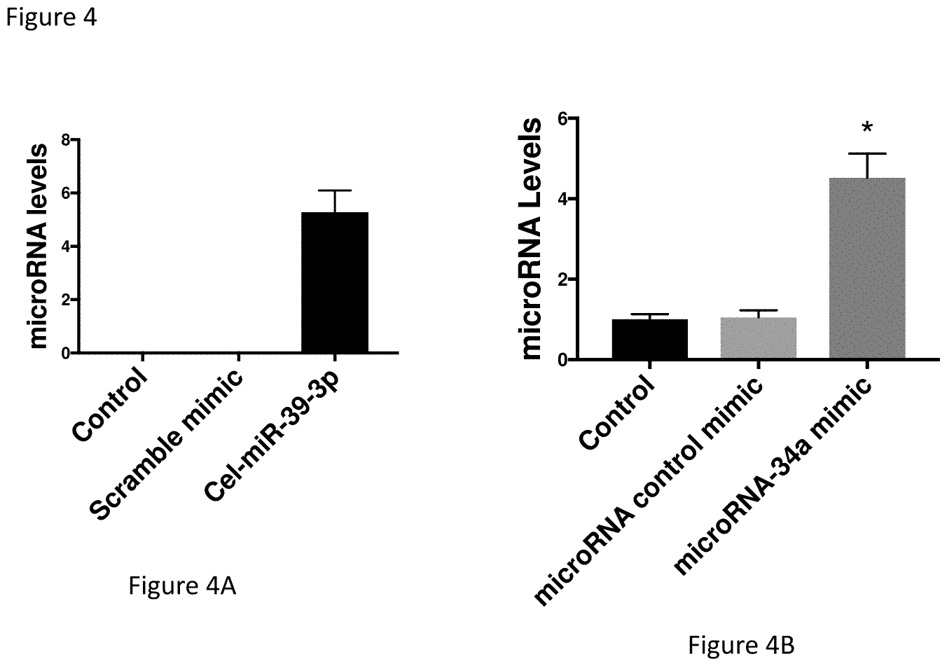

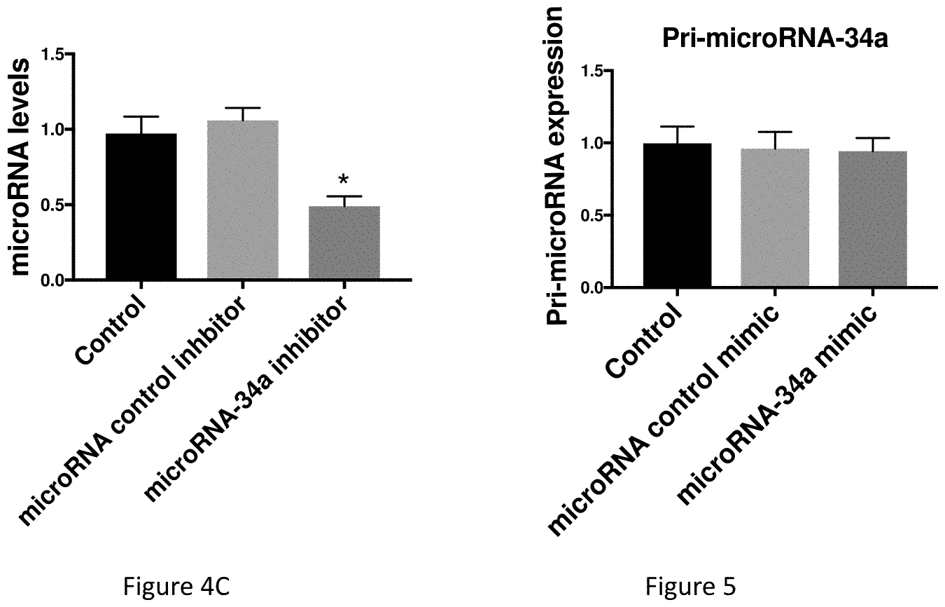

Levels of Cel-miR-39 and microRNA-34a were determined by quantitative real-time polymerase chain reaction and the results showed that the synthetic microRNA, microRNA mimic, and microRNA inhibitor were efficiently delivered to cells by engineered exosomes (FIG. 4 A-C). There was no increase in the levels of pri-microRNA-34a (primary transcripts of microRNA-34a) in the recipient cells, indicating active transfer of exosomes rather than induction of expression (FIG. 5).

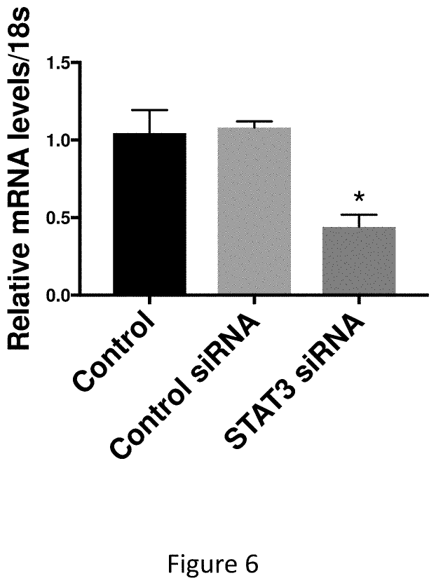

Engineered exosomes were then loaded with small interfering RNAs (siRNA) against STAT3 and co-cultured with HCC827 cells as described in Example 1. Level of STAT3 was determined by quantitative real-time polymerase chain reaction 36 h after co-culture and the results showed efficient delivery of STAT3 siRNA with engineered exosomes (FIG. 6).

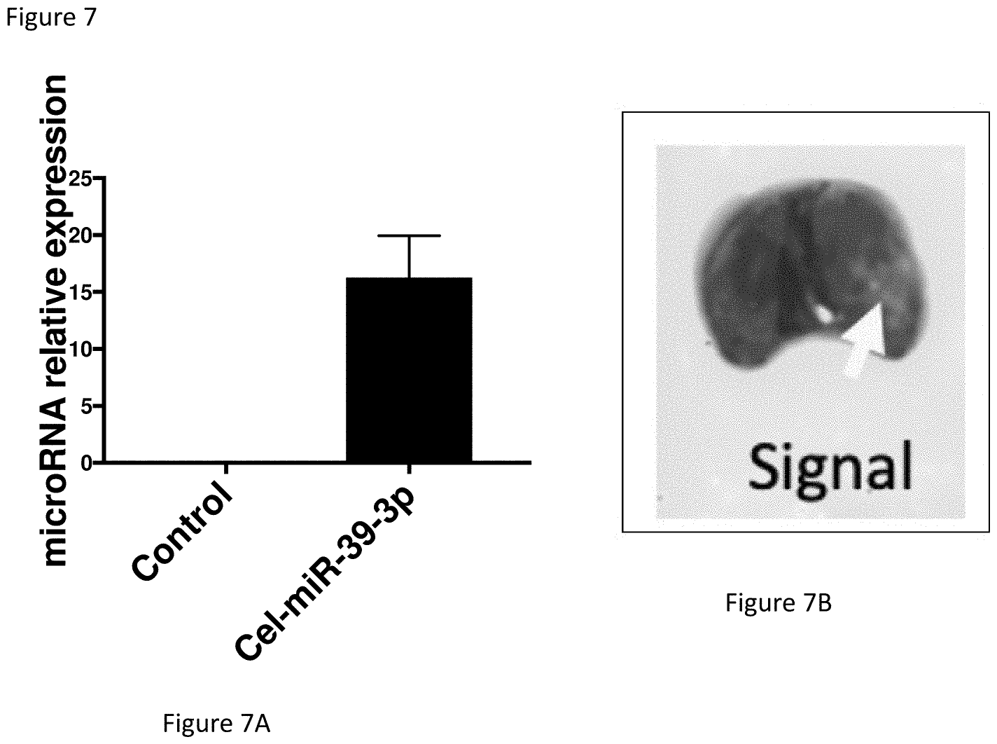

For in vivo delivery, exosomes were loaded with a synthetic version of the C. elegans microRNA, Cel-miR-39-3p, and injected to mice (IV) (FIG. 7A). Consistently, fluorescently-labeled targeted exosomes as described in Example 3 showed uptake in lung 5 minutes after IV injection identified by fluorescent imaging (FIG. 7B).

### Example 5—Engineered Exosomes Loaded with CRISPR/CAS Machinery

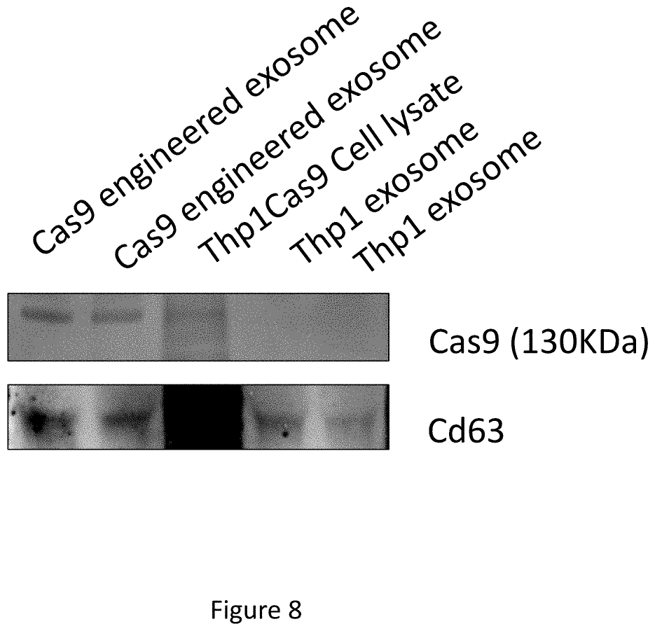

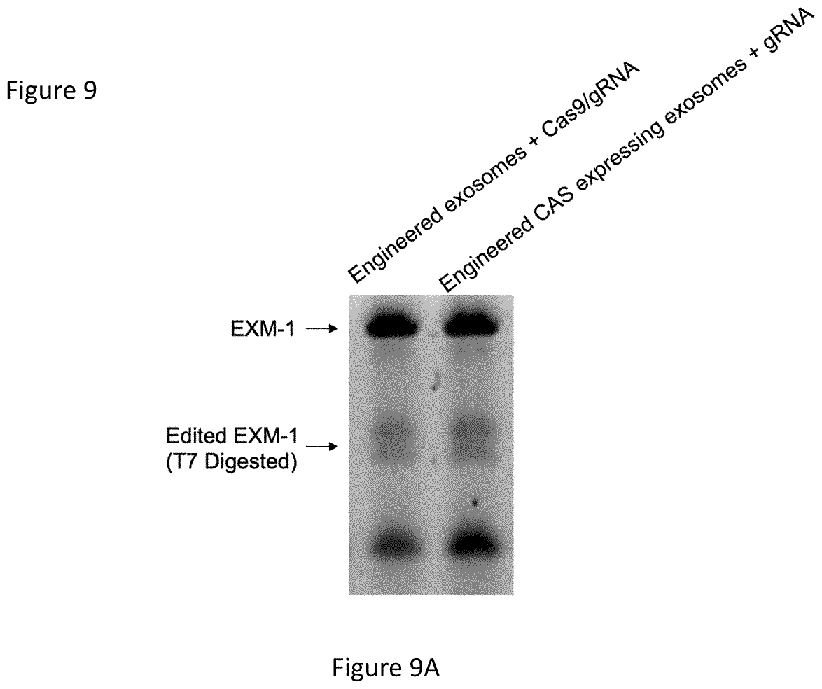

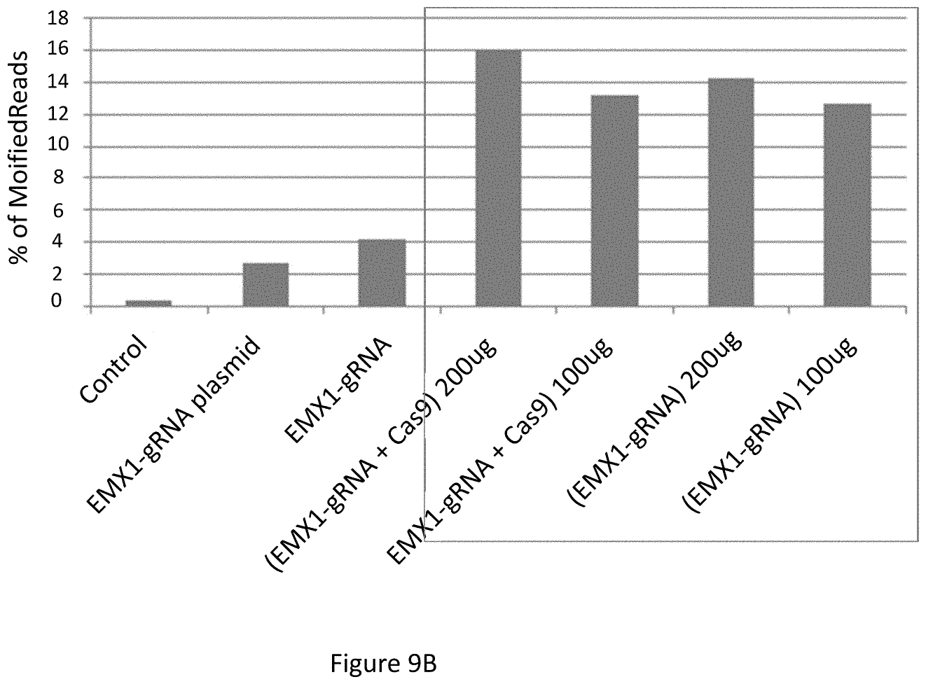

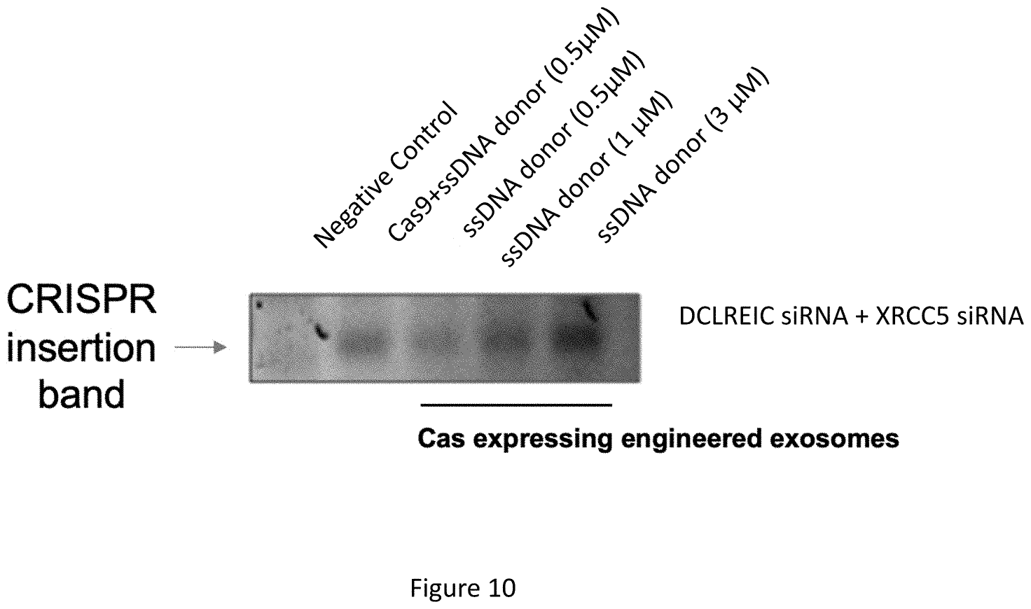

A stable engineered Cas9 expressing THP1 cell line was engineered. The cell line packaged active Cas9 into the exosomes (CASexo) (FIG. 8). Engineered CAS expressing exosomes (CASexo) were capable of producing actively editing genes and producing indels as verified by a mismatch assay (T7 endonuclease assay) (FIG. 9A). Groups that included exosomes for delivery of CRISP/CAS in the form of gRNA loaded into Cas expression exosomes or loading of gRNA/CAS complex into the exosomes showed significantly higher efficiency of editing compared to the method of plasmid DNA (FIG. 9B). Engineered exosomes showed high efficiency and dose-response relationship in mediating gene insertion through homology directed repair using CRISPR/CAS machinery (FIG. 10).

### Example 6—Engineered Exosomes are Safe and do not Cause Unwanted Side Effects

To check for the safety of engineered exosomes mice were injected with following exosomes: microRNA loaded exosomes; gRNA loaded exosomes; single-strand DNA loaded exosomes; scramble loaded exosomes; fluorescently labeled exosomes; or synthetic Cel-39 loaded exosomes (n=12).

Prior to injection into recipient mice, the exosomes were resuspended in PBS and were brought to room temperature and vortexed, 100-150 μl exosomes were injected into recipient mice (intravenous [i.v.]) at therapeutic dose of 4 mg/kg exosomal protein, three doses, 48 h apart.

Blood was drawn and animals were perfused using standardized laboratory protocol. To rule out any blood contamination, red blood lysis buffer was used as per manufacturer's instructions. and blood chemistry analysis performed. The results showed no toxicity (Table 1).

Spleen immune profiling was also performed and no change was found in immune cells. There was no significant difference in the well-being and weight of mice 4-weeks after injection of engineered exosomes.

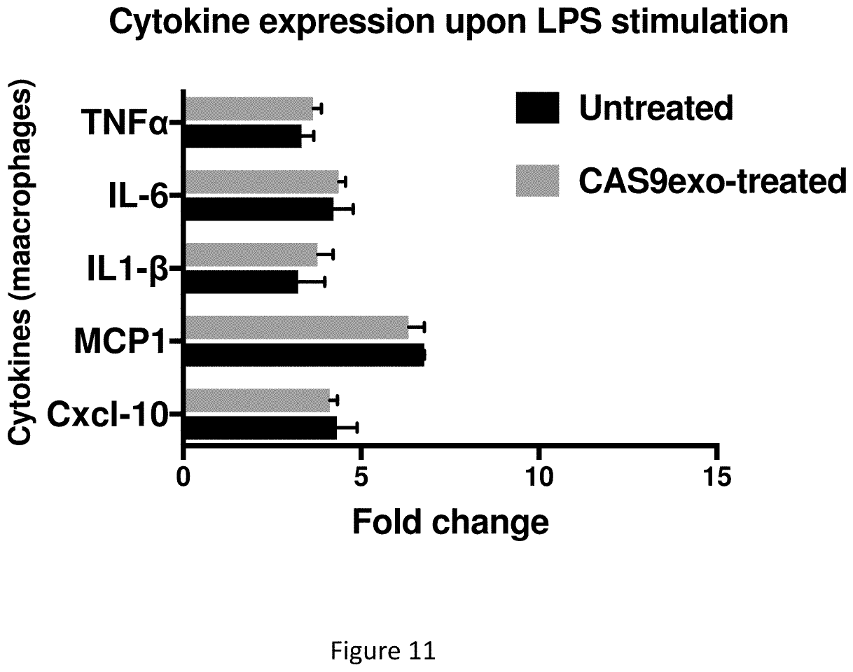

To rule out the potential of the empty engineered exosomes to induce any specific immune modulation, the engineered exosomes loaded with Cas9 (CASexo) were co-cultured with human primary macrophages. Quantitative real-time PCR showed no differential expression of cytokines after 24 hours, indicating the inert biological characteristics of engineered empty exosomes (FIG. 11).

TABLE 1
Mice blood chemistry

| | No | | | Loaded |
|---|---|---|---|---|
| Parameter | injection | PBS | Exosomes | exosomes |
| ALB (g/dL) | 3.5 ± 0.1 | 3.3 ± 0.1 | 3.6 ± 0.2 | 3.5 ± 0.3 |
| AST (U/L) | 139.2 ± 37.4 | 136.1 ± 42.1 | 137.5 ± 46.3 | 138.1 ± 37.2 |
| ALT (U/L) | 46.1 ± 12.3 | 45.7 ± 10.1 | 47.2 ± 11.3 | 46.3 ± 9.8 |
| CREAT | 0.2 ± 0.04 | 0.2 ± 0.05 | 0.2 ± 0.08 | 0.2 ± 0.04 |
| (mg/dL) | | | | |
| BUN | 18.2 ± 1.6 | 19.7 ± 2.2 | 23.8 ± 3.2 | 21.2 ± 2.0 |
| (mg/dL) | | | | |
| ALP (U/L) | 134.5 ± 12.1 | 134.5 ± 11.2 | 138.6 ± 12.1 | 135.3 ± 11.9 |
| CHOL | 83.9 ± 9.1 | 84.1 ± 10.0 | 85.3 ± 9.1 | 86.5 ± 11.4 |
| (mg/dL) | | | | |

### Example 7—In Vivo Editing of cftr1^{m1Unc} in the Lung of Cystic Fibrosis Mouse Model

Cystic fibrosis (CS) is a life-limiting autosomal recessive disorder caused by disruption of transmembrane conductance regulator (CFTR) protein (Cutting 2015). CFTR functions as a chloride channel regulated by cyclic AMP (cAMP)-dependent phosphorylation. Loss of function mutation in CFTR leads to abnormally viscous secretions in the airways of the lungs and in the ducts of the pancreas in individuals with cystic fibrosis which cause obstructions that lead to inflammation, tissue damage and destruction of both organ systems. CFTR^{tm1Unc} on C57BL/6J background mice recapitulate many phenotypic characteristics of CS in humans such as lung disease and intestinal obstructions (Kent et al. 1997). Successful editing of this mutation can lead to cure of the disease. We therefore reasoned that CASexo could represent a non-invasive and efficient method to correct CFTR.

The engineered exosome of Example 5 is injected into the CFTR^{tm1Unc} on C57BL/6J background mice. This engineered exosome has a CRISPR/Cas system for HDR directed against the 10th exon of CFTR, which targets a sequence corresponding to codon 489 of the encoded protein to replace a stop codon at position 489 which is produced from this mutant. In addition to gRNA, a single stranded DNA donor oligos of 20 nucleotides is introduced into the engineered exosome. The gRNA is designed within 10 nucleotides of HDR. To optimize the design the DNA region, 20 nt of upstream and downstream from the desired insertion site is assessed for PAM sites on both strands, and 3-5 candidate gRNAs are tested for cutting efficiency with a DNA mismatch detection assay. gRNAs with the highest possible DNA cutting efficiency are chosen. Additionally, a 30-40 nt for both 5′ and 3′ homology arms is used and the DNA oligo is modified by the addition of two phosphonothioates on each end of the DNA oligo to increase HDR efficacy.

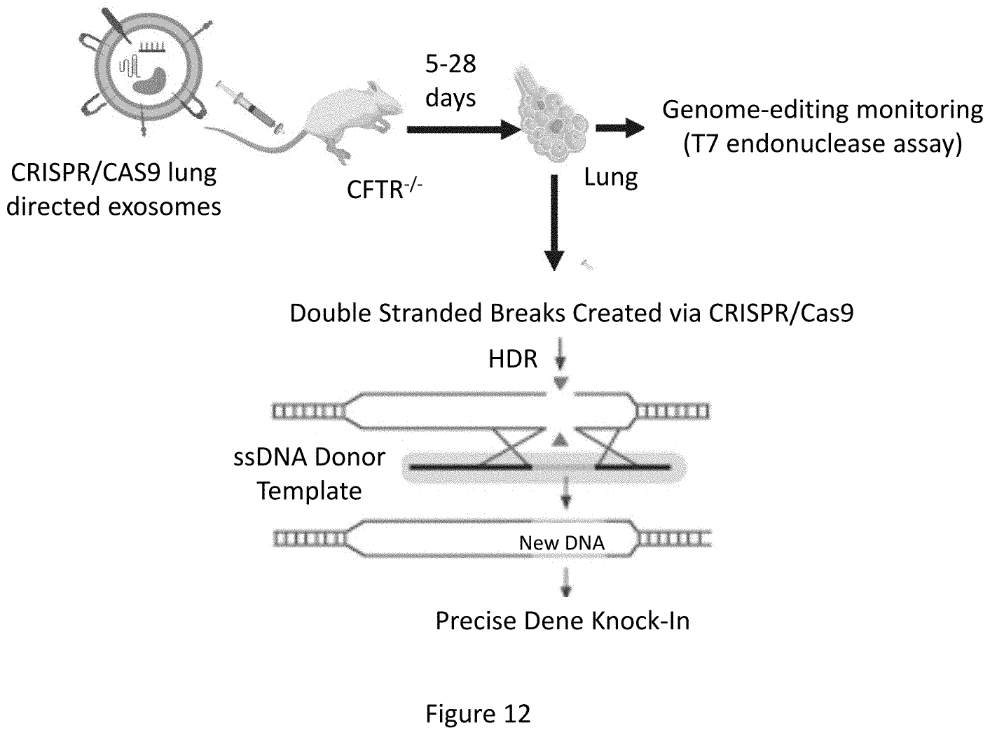

Mice are injected with the engineered exosome comprising the CRISPR/Cas system as well as the lung specific integrins as shown in Example 2. gRNA directed toward human EMX1 serve as controls. In different intervals after IV injection of the engineered exosomes, editing efficacy is quantified in the lung (5 days, 7 days, 14 days, 21 days, 28 days; n=60) by a T7 endonuclease assay, sanger sequencing, and single cell RNA sequencing after preparation of single cell suspension from the adult mouse lung. The schematic of experiment is shown in FIG. 12.

It is expected to observe editing in lungs of treated mice. Additionally, it is expected that there will be no off target editing in other organs which exosomes are not targeted toward such as spleen and liver.

### Example 8—Engineered Exosomes for the Treatment of Lung Cancers with KRAS Mutation

Kristen Rat Sarcoma viral oncogene (KRAS) is an oncogene, a member of the RAS family of membrane-associated G proteins, and encodes for a protein with intrinsic GTPase activity, which is involved in a variety of cellular responses including proliferation, cytoskeletal reorganization, and survival. KRAS acts downstream of a number of tyrosine kinases receptors, including EGFR, and is associated with activation of the RAS/RAF/MAP kinase kinase (MEK)/extracellular signal-regulated kinase (ERK) and RAS/MAPK signaling pathways. KRAS is a driver mutation occur in 25% to 35% of patients with non-small cell lung cancer (NSCLC), principally adenocarcinomas with a solid pattern.

In most cases, mutations are in the form of single-nucleotide missense variants in codons 12 and 13 (G12D, G12V, G12C). The KRAS G12C variant, with position-12 glycine substituted to a cysteine, arises from the single-nucleotide missense mutation c.34 G>T. The mutated form of KRAS is constantly activated, sending growth and proliferation signals to tumor cells. The presence of the KRAS mutation is associated with unfavorable outcome and could be a negative predictor of responsiveness to chemotherapy and is a predictor of resistance to targeted therapy with EGFR-tyrosine kinase inhibitors in NSCLC.

Exosomes substantially devoid of endogenous nucleic acids and expressing α6β4 integrin as described in Examples 2 and 3, are loaded with KRAS prime editing machinery (prime editor Cas+pegRNA). The loaded exosomes are co-cultured with KRAS 12C mutant cell line (KRAS 4B G12C) and control cell line (KRAS 4B wild type) and the editing efficiency assessed by a T7 endonuclease assay, PCR, and sequencing. The editing efficiency of exosomes loaded with the pegRNA, DNA transfection, and ribonucleoprotein delivery at the on-target site as well as off-target sites as controls is assessed.

Kras^{LSL-G12C} mice that carry a point mutation (G12C), are homozygous for the mutation and develop non-small cell lung cancer are used (O'Hagan and Heyer 2011). After 12 weeks (early treatment group) and 16 weeks (late treatment group) of Cre-mediated recombination, mice are injected intravenously (IV) with the exosomes loaded with pegRNA directed toward correcting the KRAS G12C mutation (IV). gRNA directed toward human EMX1 will serve as controls. The editing efficacy is quantified in the lung (5 days, 7 days, and 21 days n=36) after injection by a T7 endonuclease assay, Sanger sequencing, and single-cell RNA sequencing after preparation of a single-cell suspension from the adult mouse lung. Spleen and liver tissue are collected to assess non-specific off target editing in other organs by DNA sequencing.

A separate experiment is conducted to identify the efficacy of the exosomes loaded with prime editing machinery in survival outcomes and tumor burden in NSCLC (n=16). A group of untreated (liposome injected), small molecule for KRAS inhibitor-treated (SAH-SOS1A), and exosomes with gRNA targeted toward human EMX1 NSCLC mice are used as controls (n=12 per group). Micro-CT imaging at regular intervals is used to detect tumor growth and volume compared to the controls. Parenchymal and extra-parenchymal metastases is monitored by micro-CT. Survival is compared between the groups. Histopathological analysis and counting of lung tumors is performed to compare the groups at the end of the study.

Successful correction of the KRAS mutation in vitro and in the lungs of mice treated with the loaded exosome is observed. A higher survival rate and a lower tumor burden in the exosome treated group of mice is also observed. No non-detectable editing and less off target effects are observed in the exosome treated group of mice.

To further increase the editing efficiency of prime editing, a nuclear localization sequence (NLS) is covalently conjugated to the sgRNA for increased delivery to the nucleolus. NLS is an amino acid sequence that tags a protein for import into the cell nucleolus by nuclear transport. The Chelsky sequence motif of K-K/R-x-K/R(Lys-Lys/Arg-x-Lys/Arg) is used which is a monopartite NLS and will lead to nuclear localization of pegRNA via importin a (Chelsky et al. 1989; Kosugi et al. 2009).

### REFERENCES

Banaszynski et al. A rapid, reversible, and tunable method to regulate protein function in living cells using synthetic small molecules. Cell 2006; 126(5): 995-1004.
Bukong et al. Exosomes from hepatitis C infected patients transmit HCV infection and contain replication competent viral RNA in complex with Ago2-miR122-HSP90. PLoS Pathog 2014; 10(10): e1004424.
Chelsky et al. Sequence requirements for synthetic peptide-mediated translocation to the nucleus. Mol Cell Biol. 1989; 9(6): 2487-92.
Cong et al. Multiplex genome engineering using CRISPR/Cas systems. Science 2013; 339(6121): 819-23.
Han et al. The Drosha-DGCR8 complex in primary microRNA processing. Genes Dev 2004; 18(24): 3016-27.
Hood. Post isolation modification of exosomes for nanomedicine applications. Nanomedicine (Lond) 2016; 11(13): 1745-56.
Hoshino et al. Tumour exosome integrins determine organotropic metastasis. Nature 2015; 527(7578): 329-35.
Iavello et al. Role of Alix in miRNA packaging during extracellular vesicle biogenesis. Int J Mol Med 2016; 37(4): 958-66.
Jinek et al. A programmable dual-RNA-guided DNA endonuclease in adaptive bacterial immunity. Science 2012; 337(6096): 816-21.
Kick et al. CRISPR-Cas9: From a bacterial immune system to genome-edited human cells in clinical trials. Bioengineered 2017; 8(3): 280-6.
Kosugi et al. Six classes of nuclear localization signals specific to different binding grooves of importin alpha. J Biol Chem 2009; 284(1): 478-85.
Lotvall et al. Minimal experimental requirements for definition of extracellular vesicles and their functions: a position statement from the International Society for Extracellular Vesicles. J Extracell Vesicles 2014; 3: 26913.
Malhotra et al. Exosomes: Tunable Nano Vehicles for Macromolecular Delivery of Transferrin and Lactoferrin to Specific Intracellular Compartment. J Biomed Nanotechnol 2016; 12(5): 1101-14.
Momen-Heravi et al. Current methods for the isolation of extracellular vesicles. Biol Chem 2013; 394(10): 1253-62.
Momen-Heravi et al. Extracellular vesicles and their nucleic acids for biomarker discovery. Pharmacol Ther 2018; 192: 170-87.
Momen-Heravi et al. Exosome-mediated delivery of functionally active miRNA-155 inhibitor to macrophages. Nanomedicine 2014; 10(7): 1517-27.
Mout et al. In Vivo Delivery of CRISPR/Cas9 for Therapeutic Gene Editing: Progress and Challenges. Bioconjug Chem 2017; 28(4): 880-4.
O'Hagan and Heyer KRAS mouse models: modeling cancer harboring KRAS mutations. Genes and Cancer 2011; 2(3): 335-43.
Peer et al. Nanocarriers as an emerging platform for cancer therapy. Nat Nanotechnol 2007; 2(12): 751-60.
Shimaoka et al. Connexins and Integrins in Exosomes. Cancers (Basel) 2019; 11(1).
Thery et al. Exosomes: composition, biogenesis and function. Nat Rev Immunol 2002; 2(8): 569-79.
Wang et al. Comparison of adenoviral and adeno-associated viral vectors for pancreatic gene delivery in vivo. Hum Gene Ther 2004; 15(4): 405-13.
Wilbie et al. Delivery Aspects of CRISPR/Cas for in Vivo Genome Editing. Acc Chem Res 2019; 52(6): 1555-64.
Villarroya-Beltri et al. Sumoylated hnRNPA2B1 controls the sorting of miRNAs into exosomes through binding to specific motifs. Nat Commun 2013; 4: 2980.

1. An engineered exosome or extracellular vesicle, wherein the exosome or extracellular vesicle is substantially devoid of endogenous nucleic acids.

2. The engineered exosome or extracellular vesicle of claim 1, wherein the exosome or extracellular vesicle is engineered to be substantially devoid of endogenous nucleic acids by downregulating or inhibiting at least one protein which is involved in sorting or loading nucleic acids into exosomes or extracellular vesicles, wherein the downregulating or inhibiting is done using RNAi or genetic engineering.

3. The engineered exosome or extracellular vesicle of claim 2, wherein the at least one protein is chosen from the group consisting of heterogeneous nuclear ribonucleoprotein A2B1 (hnRNPA2B1), Dorsha, Alix, major vault protein (MVP), Exportin 1 and Exportin 5.

4. The engineered exosome or extracellular vesicle of claim 1, further comprising one or more of the following: (a) at least one targeting moiety or therapeutic molecule expressed on a surface of the exosome or extracellular vesicle; and (b) at least one cargo or payload.

5. The engineered exosome or extracellular vesicle of claim 4, wherein the exosome or extracellular vesicle is derived from bone marrow, red blood cells, epithelial cells, tumor cells, immune cells, or stem cells.

6. The engineered exosome or extracellular vesicle of claim 4, wherein the targeting moiety targets tissue chosen from the group consisting of lung tissue, spleen tissue, digestive organ tissue, liver tissue, kidney tissue and brain tissue.

7. The engineered exosome or extracellular vesicle of claim 4, wherein the targeting moiety targets a cell chosen from the group consisting of epithelial cells, tumor cells, fibroblast, monocytes, macrophages, dendritic cells, natural killer cells, T cells, and B cells.

8. The engineered exosome or extracellular vesicle of claim 4, wherein the targeting moiety comprises an integrin, a laminin, an antibody or an antibody fragment, a receptor, a peptide, a component of extracellular matrix, or combinations thereof.

9. The engineered exosome or extracellular vesicle of claim 4, wherein the targeting moiety or therapeutic molecule comprises a fusion protein comprising the targeting moiety or therapeutic molecule fused with at least a portion of an exosomal surface marker chosen from the group consisting of CD63, CD81, and CD9.

10. The engineered exosome or extracellular vesicle of claim 8, wherein the integrin is chosen from the group consisting of an αvβ5 integrin, an α6β4 integrin, an α6β1 integrin, and combinations thereof.

11. The engineered exosome or extracellular vesicle of claim 4, wherein the cargo or payload comprises a nucleic acid, a protein, a polypeptide, a small molecule or combinations thereof.

12. The engineered exosome or extracellular vesicle of claim 11, wherein the cargo or payload is a nucleic acid chosen from the group consisting of a single-stranded DNA (ssDNA), a double-stranded DNA (dsDNA), a donor/template DNA, s cDNA. a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof.

13. The engineered exosome or extracellular vesicle of claim 4, wherein the cargo or payload is a Clustered Regularly Interspaced Short Palindromic Repeats (CRISPR)-CRISPR associated (Cas) (CRISPR-Cas) system.

14. The engineered exosome or extracellular vesicle of claim 13, further comprising a guide RNA (gRNA) or a prime editing RNA (pegRNA).

15. The engineered exosome or extracellular vesicle of claim 14, wherein the gRNA forms a Cas/gRNA ribonucleoprotein complex with a Cas protein.

16. The engineered exosome or extracellular vesicle of claim 13, wherein the CRISPR-Cas system comprises Cas9.

17. The engineered exosome or extracellular vesicle of claim 13, wherein the CRISPR-Cas system comprises CAS13a, CAS13b, CAS13c, CAS13d, Cas12a, SiT-Cas12a, Cpf1, C2c1, C2c2, or C2c3 or dCas9 or any of those CAS proteins fused to other proteins.

18. The engineered exosome or extracellular vesicle of claim 14, wherein the gRNA or pegRNA targets a sequence corresponding to KRAS mutations chosen from the group consisting of G12C, G12V, and G12D.

19. The engineered exosome or extracellular vesicle of claim 18, further comprising a donor DNA targeted to correct the KRAS mutation by directed homology repair, wherein the donor DNA comprises a single stranded DNA of about 20 to about 100 nucleotides.

20. A method of treating a disease, comprising administering a therapeutically effective amount of the engineered exosome or extracellular vesicle of claim 4, wherein the disease is chosen from the group consisting of cystic fibrosis, genetic diseases, autoimmune diseases, inflammatory diseases, and cancer.
